SEQUENCE LISTING (2 sequences)

SEQ ID NO: 1 - DNA, 19 bp, synthetic construct
  misc_feature 1..19: note = Description of Artificial Sequence: Synthetic oligonucleotide
  source 1..19: mol_type = other DNA
taatacgactcactatagg

SEQ ID NO: 2 - DNA, 48 bp, synthetic construct
  misc_feature 1..48: note = Description of Artificial Sequence: Synthetic oligonucleotide
  source 1..48: mol_type = other DNA
ctagcataaccccttggggcctctaaacgggtcttgaggggttttttg

(19) United States
(12) Patent Application Publication
(10) Pub. No.: US 2024/0254535 A1
(43) Pub. Date: Aug. 1, 2024

(54) SYSTEMS AND METHODS FOR CELL-FREE POLYPEPTIDE SYNTHESIS

(71) Applicant: National Resilience, Inc., San Diego, CA (US)

(72) Inventors: Danielle Jessica Yoesep, San Francisco, CA (US); Weston Kevin Kightlinger, San Francisco, CA (US); Sajita Caroni Shah-Morales, San Francisco, CA (US)

(21) Appl. No.: 18/473,087

(22) Filed: Sep. 22, 2023

Related U.S. Application Data
(63) Continuation of application No. PCT/US2022/021651, filed on Mar. 24, 2022.
(60) Provisional application No. 63/165,335, filed on Mar. 24, 2021.

(51) Int. Cl. C12P 21/02; C12N 15/10

(52) U.S. Cl. CPC C12P 21/02; C12N 15/1017

(57) ABSTRACT

Disclosed herein is a method for cell-free polypeptide synthesis. The method comprises lysing a cell to produce a cellular lysate, passing the cellular lysate through a filter to produce a cellular extract, contacting the cellular extract with a nucleic acid encoding a polypeptide to produce a reaction mixture, and incubating the reaction mixture to produce the polypeptide.

### CROSS-REFERENCE TO RELATED APPLICATIONS

This application is a continuation of International Application No. PCT/US2022/021651, filed on Mar. 24, 2024, which claims the benefit of U.S. Provisional Application No. 63/165,335 filed on Mar. 24, 2021, all of which are incorporated herein by reference in their entireties.

### SEQUENCE LISTING

The instant application contains a Sequence Listing which has been submitting electronically in XML format and is hereby incorporated by reference in its entirety. Said XML copy, created Sep. 19, 2023, is named “62633-704_301-Sequence-Listing.xml” and is 3,771 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Disclosed herein, in some embodiments, is a method for cell-free polypeptide synthesis. In some embodiments, the method comprises lysing a cell to produce a cellular lysate, passing the cellular lysate through a filter to produce a cellular extract, contacting the cellular extract with a nucleic acid encoding a polypeptide to produce a reaction mixture, and incubating the reaction mixture to produce the polypeptide. In some embodiments, the cellular lysate is not subject to clarification through centrifugation. In some embodiments, the cellular extract is not subject to clarification through centrifugation. In some embodiments, the reaction mixture is not subject to clarification through centrifugation. In some embodiments, the method comprises flocculating the cellular lysate before the passing through the filter. In some embodiments, the method comprises precipitating the cellular lysate before the passing through the filter. In some embodiments, the filter comprises a surface filter. In some embodiments, the filter comprises a depth filter. In some embodiments, the passing comprises one or more selected from the group comprising cross-flow filtration, belt filtration, microfiltration, ultrafiltration, microporous filtration, vacuum-drum filtration, sieving, membrane filtration, sand filtration, and screen filtration. In some embodiments, the filter comprises a pore that retains or rejects a particle with a size of more than about 100 microns. In some embodiments, the filter comprises a pore that retains or rejects a particle with a size of more than about 15 microns. In some embodiments, the filter comprises a pore that retains or rejects a particle with a size of more than about 5 microns. In some embodiments, the filter comprises a pore that retains or rejects a particle with a size of more than about 0.4 micron. In some embodiments, the filter comprises a pore that retains or rejects a particle with a size of more than about 0.2 micron. In some embodiments, the filter comprises a pore that retains or rejects a particle with a size of more than about 0.1 micron. In some embodiments, the filter comprises a pore that retains or rejects a particle with a size of less than 100 microns. In some embodiments, the filter comprises a pore that retains or rejects a particle with a size of less than 15 microns. In some embodiments, the filter comprises a pore that retains or rejects a particle with a size of less than 5 microns. In some embodiments, the filter comprises a pore that retains or rejects a particle with a size of less than 0.4 micron. In some embodiments, the filter comprises a pore that retains or rejects a particle with a size of less than 0.2 micron. In some embodiments, the filter comprises a pore that retains or rejects a particle with a size of less than 0.1 micron. In some embodiments, the filter comprises a pore that retains or rejects a particle with a size between about 0.4 micro and about 15 microns. In some embodiments, the filter comprises a pore that retains or rejects a particle with a size between about 0.2 micro and about 0.4 micron. In some embodiments, the filter comprises a pore that retains or rejects a particle with a size of about 0.2 micron. In some embodiments, the filter comprises at least two filters that are arranged in series or in parallel. In some embodiments, the filter comprises at least two filters that are arranged in series with gradually decreasing pore sizes. In some embodiments, the filter comprises a first filter comprising a pore that retains or rejects a particle with a size between about 0.5 micron and about 15 microns, a second filter comprising a pore that retains or rejects a particle with a size between about 0.2 micron and about 0.5 micron, and a third filter comprising a pore that retains or rejects a particle with a size of about 0.2 micron. In some embodiments, the depth filter comprises a medium. In some embodiments, the medium forms a flow channel. In some embodiments, the medium comprises two fibers that are bonded or fixed to provide a matrix. In some embodiments, one of the two fibers is a cellulose fiber. In some embodiments, the medium is positively charged. In some embodiments, the medium is neutrally charged. In some embodiments, the medium is negatively charged. In some embodiments, the depth filter comprises a filter aid. In some embodiments, the depth filter comprises a pore that retains a particle with a size that is either larger or smaller than the size of the pore. In some embodiments, the depth filter comprises a single-use filter. In some embodiments, the depth filter comprises a lenticular filter, a deep bed filter, a pad filter, a panel filter, a cartridge-type depth filter, or a combination thereof. In some embodiments, the filter comprises a first depth filter comprising a pore that retains or rejects a particle with a size between about 0.5 micron and about 15 microns, a second depth filter comprising a pore that retains or rejects a particle with a size between about 0.2 micron and about 0.5 micron, and a third filter comprising a sterilization-grade membrane and a pore that retains or reject a particle with a size of about 0.2 micron. In some embodiments, the cellular lysate is subject to filtration through the first depth filter, then, the second depth filter, and, last, the third filter. In some embodiments, the method further comprises obtaining a cell from a cell culture. In some embodiments, the obtaining comprises subjecting the cell culture to one or more selected from the group comprising centrifugation, tangential flow filtration, membrane separation, and a combination thereof. In some embodiments, the membrane separation comprises hollow fiber membrane separation. In some embodiments, the lysing comprises one or more selected from the group comprising sonication, homogenization, nitrogen cavitation, freeze-thawing, French press lysis, bead beating, syringing, chemical lysis, enzymatic lysis, osmotic lysis, and a combination thereof. In some embodiments, the method further comprises freezing the cellular extract. In some embodiments, the method further comprises freeze-drying the cellular extract. In some embodiments, the cell comprises a transgenic cell, a mammalian cell, a bacterial cell, a plant cell, a yeast cell, an insect cell, a fungal cell, an algal cell, or a combination thereof. In some embodiments, the nucleic acid comprises a DNA. In some embodiments, the DNA comprises an open reading frame of the polypeptide. In some embodiments, the DNA comprises a promoter for a DNA-dependent RNA polymerase, wherein the open reading frame is operatively linked to the promoter. In some embodiments, the DNA comprises a ribosome binding site located at the 5′ end of the opening reading frame. In some embodiments, the DNA comprises a terminator located at the 3′ end of the open reading frame. In some embodiments, the promoter comprises a T7 promoter. In some embodiments, the T7 promoter comprises a sequence having at least 80% sequence identity with 5′-TAATACGACTCACTATAGG-3′ (SEQ ID No: 1). In some embodiments, the T7 promoter comprises a sequence having at least 85% sequence identity with SEQ ID No: 1. In some embodiments, the T7 promoter comprises a sequence having at least 90% sequence identity with SEQ ID No: 1. In some embodiments, the T7 promoter comprises a sequence having at least 95% sequence identity with SEQ ID No: 1. In some embodiments, the T7 promoter comprises SEQ ID No: 1. In some embodiments, the terminator comprises a T7 terminator. In some embodiments, the T7 terminator comprises a sequence having at least 80% sequence identity with 5′-CTAGCATAACCCCTTGGGGCCTCTAAACGGGTCTTGAGGGGTTTTTTG-3′ (SEQ ID No: 2). In some embodiments, the T7 terminator comprises a sequence having at least 85% sequence identity with SEQ ID No: 2. In some embodiments, the T7 terminator comprises a sequence having at least 90% sequence identity with SEQ ID No: 2. In some embodiments, the T7 terminator comprises a sequence having at least 95% sequence identity with SEQ ID No: 2. In some embodiments, the T7 terminator comprises SEQ ID No: 2. In some embodiments, the reaction mixture comprises a divalent cation. In some embodiments, the passing is carried out at a temperature between about 1° C. and about 40° C. In some embodiments, the reaction mixture comprises an amino acid, a nucleotide, a salt, a cofactor, an energy source, a translation template, or a combination thereof. In some embodiments, the contacting comprises adding to the cellular extract the nucleic acid encoding the polypeptide and one or more selected from the amino acid, the nucleotide, the salt, the cofactor, the energy source, the translation template, or a combination thereof. In some embodiments, the energy source comprises a phosphate group. In some embodiments, the energy source comprises a non-phosphorylated energy group. In some embodiments, the energy source comprises glutamate and is present at a concentration between about 10 mM and about 400 mM. In some embodiments, the energy source comprises pyruvate and is present at a concentration between about 10 mM and about 400 mM. In some embodiments, the energy source comprises glucose at a concentration between about 10 and about 400 mM. In some embodiments, the salt comprises potassium at a concentration between about 50 mM and 500 mM. In some embodiments, the salt comprises magnesium at a concentration between about 1 mM and 30 mM. In some embodiments, the salt comprises potassium at a concentration between about 10 mM and about 400 mM and magnesium at a concentration between about 1 mM and 30 mM. In some embodiments, the salt comprises ammonium at a concentration between about 1 mM and about 400 mM. In some embodiments, the method further comprises isolating the polypeptide from the reaction mixture. In some embodiments, the isolating is carried out at a temperature between about 1° C. and 30° C. In some embodiments, the nucleotide comprises a nucleoside monophosphate (NMP), a nucleoside diphosphate (NDP), a nucleoside triphosphate (NTP), or a combination thereof. In some embodiments, the reaction mixture comprises a transfer RNA.

### BRIEF DESCRIPTION OF THE DRAWINGS

The novel features of the disclosure are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 compares a state-of-the-art method for clarification of cellular lysate via centrifugation (upper) with a method for clarification of cellular lysate via filtration (lower).

FIG. 2 illustrates a multi-step filtration process for clarification of cellular lysate.

FIG. 3 illustrates a state-of-the-art cell-free polypeptide synthesis method using cellular lysate clarified via centrifugation (left) and a cell-free polypeptide synthesis method using cellular lysate clarified via filtration (right).

FIG. 4 illustrates the yield of a super-folder green fluorescent protein (sfGFP) produced from cell-free polypeptide synthesis methods using cellular lysate clarified via centrifugation (left) and cellular lysate clarified via filtration (right).

FIG. 5 illustrates the effect of different filter pore sizes on protein yield of CFPS.

FIG. 6 illustrates the effect of different filter pore sizes on throughput and average flux achieved during lysate clarification.

### DETAILED DESCRIPTION

Cell-free polypeptide synthesis (CFPS) is a platform technology that uses crude cellular extract to synthesize desired polypeptide products rapidly and reliably. This crude cellular extract is the foundation of a cell-free reaction, as it utilizes its endogenous machinery to enable polypeptide translation (and optionally, mRNA transcription) to generate polypeptide and protein products from nucleic acid templates (such as plasmid or linear DNA) as well as supplemented nucleotides, amino acids, energy sources, and other necessary cofactors. CFPS has many potential advantages over cellular expression including faster expression timelines, the freedom to customize expression conditions, and obviating the need to generate unique cell lines for each synthesized polypeptide or protein by direct addition of linear or plasmid DNA templates.

However, cell-free extract preparation methods are long and laborious. Much effort in the CFPS field has been dedicated to streamlining extract preparation, which is generally composed of cell growth, cell harvest, cell lysis, and post-lysis processing. Manufacturing-scale polypeptide or protein synthesis may use alternative methods of post-lysis processing that reduce energy costs and cleaning while maintaining high protein yields. Post-lysis clarification typically relies on centrifugation. Numerous reports describe attempts to lower the power demands of post-lysis centrifugation step (e.g., lower speed and/or fewer spins). For example, the operation time for clarifying a cellular lysate using a centrifuge can range from about 30 minutes to up to 24 hours. Additional time is required to clean the centrifugation equipment (e.g., up to 48 hours of cleaning time at scale). Depending on scale, centrifugation equipment can cost $10,000-$1,000,000 to purchase and have annual operating costs of $50,000-$200,000. Centrifugation at high speeds used for post-lysis processing generally requires expensive and reusable stainless-steel equipment that carries a risk of cross contamination and requires time-intensive cleaning validation for use on multiple products, particularly those made under GMP conditions. The need for centrifugation therefore makes post-lysis processing incompatible with disposable manufacturing methods (which are highly flexible, do not carry cross-contamination risks, and are now standard in the biopharmaceutical industry). Centrifugation can also be difficult to implement in a high-throughput format, such as a multi-well plate, for high-throughput extract generation. A process that replaces the centrifugation step with another method of clarification during the extract preparation may produce protein at a lower cost and/or less time per gram, while the titer of the protein produced using the improved extraction method may be comparable. The improved process may be implemented in resource-limited settings, for high-throughput extract generation, with less capital investment, and in existing or new disposable manufacturing settings.

Therefore, there remains a need for scaled-up CFPS systems and methods that can prepare a cellular extract efficiently and inexpensively with reduced time and equipment cost and can be implemented in resource-limited or disposable manufacturing settings.

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers+/−10% thereof, or 10% below the lower listed limit and 10% above the higher listed limit for the values listed for a range.

The term “cell lysis” refers to disruption of the cellular membrane. Lysis generally is carried out in a buffer solution configured to limit or prevent inactivation, denaturation, and degradation of cellular proteins.

The term “cellular lysate” generally includes the contents of lysed cells and the components of the lysis buffer solution. In some cases, the cellular lysate includes foreign material introduced during cell culture. The terms “cellular lysate” and “cell-free lysate” are used interchangeably in the present disclosure.

The terms “clarify”, “clarification”, “clarification step,” generally refer to one or more steps used to prepare a cellular extract for use with cell-free polypeptide synthesis (CFPS) performed to remove whole cells, cellular debris, and foreign material present in cellular lysate that are not required for cell-free polypeptide synthesis.

The term “depth filter” is a filter that entraps or absorbs particles both within and on the filter.

The term “cellular debris” refers to cellular proteins, membranes, cell wall components, and/or genomic DNA usually present in cellular lysates.

The term “cell-free extract” refers to a product obtained by clarifying a cellular lysate.

### Cell-Free Polypeptide Synthesis

Cell-free polypeptide synthesis (CFPS) is a platform technology that uses crude cellular extract to synthesize desired polypeptide or protein products rapidly and reliably. CFPS utilizes a cell's endogenous machinery and/or supplemented nucleotides, amino acids, energy sources, and other necessary cofactors, to transcribe and translate mRNA from nucleic acid templates, such as plasmid or linear DNA.

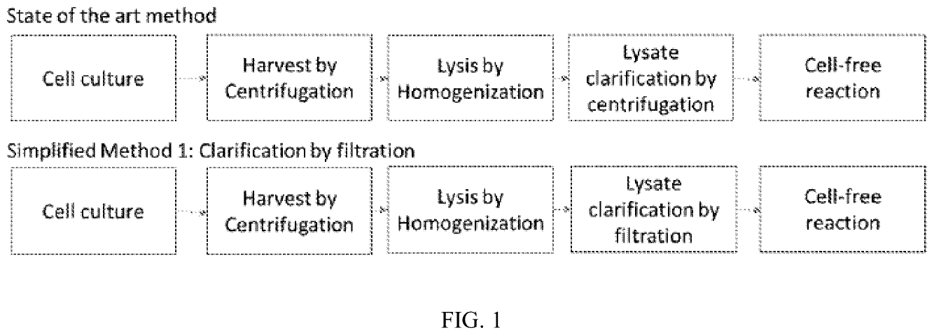

As illustrated in FIG. 1, the existing cell-free extract-based CFPS method (upper panel) comprises the following steps, cell culture or cell growth, cell harvest by centrifugation, cell lysis by homogenization, cell lysate clarification by centrifugation, and CFPS using cell-free extract clarified through centrifugation. The post-lysis clarification process usually relies on centrifugation, which may require a lengthy operation time and a substantial cost for equipment such as a centrifuge. The clarification step is intended to remove whole cells, cellular debris, and foreign material present in cellular lysate that are not needed for CFPS and may interfere with the ability of the extract to make the desired polypeptide or protein product specifically and at high yields and quality (including post-translational modification, folding, and limited proteolysis) as well as the extract's reproducibility, low bioburden, and material properties. There is a need for a post-lysis processing method that replaces the centrifugation step with one that may lower both the time and cost of CFPS and can be implemented in resource-limited or disposable manufacturing settings. Disclosed herein are methods and systems for CFPS that use cellular extract clarified through filtration.

### Methods and Systems for Clarification of Cellular Lysate Via Filtration

### Methods for Clarification of Cellular Lysate Via Filtration

As illustrated in FIG. 1, disclosed herein is a method (lower panel) for CFPS that uses filtration, instead of centrifugation, to clarify the cellular lysate to obtain cell-free extract for CFPS.

Described herein, in some embodiments, is a method for cell-free polypeptide synthesis (CFPS). In some embodiments, the method may comprise a step of obtaining a cell. In some embodiments, the obtaining may comprise growing the cell. In some embodiments, the obtaining may comprise culturing the cell. In some embodiments, the cell may comprise a cell suitable for CFPS. In some embodiments, the cell may comprise one or more selected from the group comprising a transgenic cell, a mammalian cell, a bacterial cell, a plant cell, a yeast cell, an insect cell, a fungal cell, and an algal cell. In some embodiments, the cell may comprise one or more selected from the group comprising Escherichia coli, Spodoptera frugiperda, Saccharomyces cerevisiae, Neurospora crassa, Streptomyces, Vibrio natriegens, Bacillus subtilis, Tobacco, Arabidopsis, Pseudomonas Putida, Bacillus megaterium, Archaea, Leishmania tarentolae, a Chinese hamster ovary cell, a rabbit reticulocyte, a wheat germ cell, a HEK cell, a HeLa cell, and a genetically modified variant thereof.

In some embodiments, the method may comprise a step of harvesting the cell. In some embodiments, the harvesting may comprise subjecting the cell to one or more selected from the group comprising centrifugation, tangential flow filtration, and membrane separation centrifugation. In some embodiments, the harvesting may comprise subjecting the cell in a cell culture to one or more selected from the group comprising centrifugation, tangential flow filtration, and membrane separation centrifugation. In some embodiments, the membrane separation may comprise tangential flow filtration, normal-flow filtration, or hollow fiber membrane separation. In some embodiments, the step of the harvesting may comprise resuspending the cell. In some embodiments, the cell may be resuspended in a volume between about 0.01 ml and 50 ml per wet weight gram of cells, e.g., about 0.01 ml, 0.02 ml, 0.03 ml, 0.04 ml, 0.05 ml, 0.06 ml, 0.07 ml, 0.08 ml, 0.09 ml, 0.1 ml, 0.11 ml, 0.12 ml, 0.13 ml, 0.14 ml, 0.15 ml, 0.16 ml, 0.17 ml, 0.18 ml, 0.19 ml, 0.2 ml, 0.21 ml, 0.22 ml, 0.23 ml, 0.24 ml, 0.25 ml, 0.26 ml, 0.27 ml, 0.28 ml, 0.29 ml, 0.3 ml, 0.31 ml, 0.32 ml, 0.33 ml, 0.34 ml, 0.35 ml, 0.36 ml, 0.37 ml, 0.38 ml, 0.39 ml, 0.4 ml, 0.41 ml, 0.42 ml, 0.43 ml, 0.44 ml, 0.45 ml, 0.46 ml, 0.47 ml, 0.48 ml, 0.49 ml, 0.5 ml, 0.55 ml, 0.6 ml, 0.65 ml, 0.7 ml, 0.75 ml, 0.8 ml, 0.85 ml, 0.9 ml, 0.95 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 21 ml, 22 ml, 23 ml, 24 ml, 25 ml, 26 ml, 27 ml, 28 ml, 29 ml, 30 ml, 31 ml, 32 ml, 33 ml, 34 ml, 35 ml, 36 ml, 37 ml, 38 ml, 39 ml, 40 ml, 41 ml, 42 ml, 43 ml, 44 ml, 45 ml, 46 ml, 47 ml, 48 ml, 49 ml, or about 50 ml, or any volume therebetween.

In some embodiments, the method may comprise a step of lysing the cell to produce a cellular lysate. In some embodiments, the cellular lysate is not subject to clarification through centrifugation. In some embodiments, the lysing may comprise one or more selected from the group comprising French press, bead beating, sonication, homogenization, nitrogen cavitation, freeze-thawing, syringing, chemical lysis, enzymatic lysis, and osmotic lysis.

In some embodiments, the method may comprise a step of passing the cellular lysate through a filter to produce a cellular extract. In some embodiments, the cellular lysate is not subject to clarification through centrifugation. In some embodiments, the cellular extract is not subject to clarification through centrifugation. In some embodiments, the method may comprise a step of flocculating the cellular lysate before the step of the passing through the filter. In some embodiments, the method may comprise a step of precipitating components within the cellular lysate before the step of the passing through the filter.

In some embodiments, a variety of filters may be used to produce the cellular extract from the cellular lysate through filtering or clarification. In some embodiments, the filter may comprise one filter. In some embodiments, the filter may comprise at least two filters, e.g., two filters, three filters, four filters, five filters, six filters, seven filters, eight filters, nine filters, ten filters, or more than ten filters. In some embodiments, the filter may comprise a filter disclosed herein, or a combination of two or more of the filters disclosed herein. When the filter comprises at least two filters, one of the at least two filters may comprise a filter disclosed herein. When the filter comprises at least two filters, each of the at least two filters may comprise a filter disclosed herein. In some embodiments, the at least two filters may be arranged in series. In some embodiments, the at least two filters may be arranged in series with gradually decreasing pore sizes. In some embodiments, the step of the passing of the method may comprise passing the cellular lysate through the at least two filters arranged in series to produce the cellular extract. In some embodiments, the passing may comprise passing the cellular lysate through the at least two filters arranged in series so that the cellular lysate is filtered through a series of filters with gradually decreasing pore sizes to produce the cellular extract. In some embodiments, the at least two filters may be arranged in parallel. In some embodiments, the step of the passing of the method may comprise passing the cellular lysate through at least two filters arranged in parallel to produce the cellular extract. In some embodiments, some, but not all, of the at least two filters may be arranged in series. In some embodiments, some, but not all, of the at least two filters may be arranged in parallel. In some embodiments, some of the at least two filters may be arranged in series and some of the at least two filters may be arranged in parallel.

In some embodiments, the filter may comprise a pore that retains or rejects a particle with a size between about 100 microns and about 1000 microns, e.g., about 100 microns, 110 microns, 120 microns, 130 microns, 140 microns, 150 microns, 160 microns, 170 microns, 180 microns, 190 microns, 200 microns, 210 microns, 220 microns, 230 microns, 240 microns, 250 microns, 260 microns, 270 microns, 280 microns, 290 microns, 300 microns, 310 microns, 320 microns, 330 microns, 340 microns, 350 microns, 360 microns, 370 microns, 380 microns, 390 microns, 400 microns, 410 microns, 420 microns, 430 microns, 440 microns, 450 microns, 460 microns, 470 microns, 480 microns, 490 microns, 500 microns, 510 microns, 520 microns, 530 microns, 540 microns, 550 microns, 560 microns, 570 microns, 580 microns, 590 microns, 600 microns, 610 microns, 620 microns, 630 microns, 640 microns, 650 microns, 660 microns, 670 microns, 680 microns, 690 microns, 700 microns, 710 microns, 720 microns, 730 microns, 740 microns, 750 microns, 760 microns, 770 microns, 780 microns, 790 microns, 800 microns, 810 microns, 820 microns, 830 microns, 840 microns, 850 microns, 860 microns, 870 microns, 880 microns, 890 microns, 900 microns, 910 microns, 920 microns, 930 microns, 940 microns, 950 microns, 960 microns, 970 microns, 980 microns, 990 microns, or about 1000 microns, or any size therebetween.

In some embodiments, the filter may comprise a pore that retains or rejects a particle with a size between about 100 microns and about 1000 microns. In some embodiments, the filter may comprise a pore that retains or rejects a particle with a size between about 100 microns and about 900 microns. In some embodiments, the filter may comprise a pore that retains or rejects a particle with a size between about 100 microns and about 800 microns. In some embodiments, the filter may comprise a pore that retains or rejects a particle with a size between about 100 microns and about 700 microns. In some embodiments, the filter may comprise a pore that retains or rejects a particle with a size between about 100 microns and about 600 microns. In some embodiments, the filter may comprise a pore that retains or rejects a particle with a size between about 100 microns and about 500 microns. In some embodiments, the filter may comprise a pore that retains or rejects a particle with a size between about 100 microns and about 400 microns. In some embodiments, the filter may comprise a pore that retains or rejects a particle with a size between about 100 microns and about 300 microns. In some embodiments, the filter may comprise a pore that retains or rejects a particle with a size between about 100 microns and about 200 microns. In some embodiments, the filter may comprise a pore that retains or rejects a particle with a size between about 200 microns and about 1000 microns. In some embodiments, the filter may comprise a pore that retains or rejects a particle with a size between about 200 microns and about 900 microns. In some embodiments, the filter may comprise a pore that retains or rejects a particle with a size between about 200 microns and about 800 microns. In some embodiments, the filter may comprise a pore that retains or rejects a particle with a size between about 200 microns and about 700 microns. In some embodiments, the filter may comprise a pore that retains or rejects a particle with a size between about 200 microns and about 600 microns. In some embodiments, the filter may comprise a pore that retains or rejects a particle with a size between about 200 microns and about 500 microns. In some embodiments, the filter may comprise a pore that retains or rejects a particle with a size between about 200 microns and about 400 microns. In some embodiments, the filter may comprise a pore that retains or rejects a particle with a size between about 200 microns and about 300 microns. In some embodiments, the filter may comprise a pore that retains or rejects a particle with a size between about 300 microns and about 1000 microns. In some embodiments, the filter may comprise a pore that retains or rejects a particle with a size between about 300 microns and about 900 microns. In some embodiments, the filter may comprise a pore that retains or rejects a particle with a size between about 300 microns and about 800 microns. In some embodiments, the filter may comprise a pore that retains or rejects a particle with a size between about 300 microns and about 700 microns. In some embodiments, the filter may comprise a pore that retains or rejects a particle with a size between about 300 microns and about 600 microns. In some embodiments, the filter may comprise a pore that retains or rejects a particle with a size between about 300 microns and about 500 microns. In some embodiments, the filter may comprise a pore that retains or rejects a particle with a size between about 300 microns and about 400 microns. In some embodiments, the filter may comprise a pore that retains or rejects a particle with a size between about 400 microns and about 1000 microns. In some embodiments, the filter may comprise a pore that retains or rejects a particle with a size between about 400 microns and about 900 microns. In some embodiments, the filter may comprise a pore that retains or rejects a particle with a size between about 400 microns and about 800 microns. In some embodiments, the filter may comprise a pore that retains or rejects a particle with a size between about 400 microns and about 700 microns. In some embodiments, the filter may comprise a pore that retains or rejects a particle with a size between about 400 microns and about 600 microns. In some embodiments, the filter may comprise a pore that retains or rejects a particle with a size between about 400 microns and about 500 microns. In some embodiments, the filter may comprise a pore that retains or rejects a particle with a size between about 500 microns and about 1000 microns. In some embodiments, the filter may comprise a pore that retains or rejects a particle with a size between about 500 microns and about 900 microns. In some embodiments, the filter may comprise a pore that retains or rejects a particle with a size between about 500 microns and about 800 microns. In some embodiments, the filter may comprise a pore that retains or rejects a particle with a size between about 500 microns and about 700 microns. In some embodiments, the filter may comprise a pore that retains or rejects a particle with a size between about 500 microns and about 600 microns. In some embodiments, the filter may comprise a pore that retains or rejects a particle with a size between about 600 microns and about 1000 microns. In some embodiments, the filter may comprise a pore that retains or rejects a particle with a size between about 600 microns and about 900 microns. In some embodiments, the filter may comprise a pore that retains or rejects a particle with a size between about 600 microns and about 800 microns. In some embodiments, the filter may comprise a pore that retains or rejects a particle with a size between about 600 microns and about 700 microns. In some embodiments, the filter may comprise a pore that retains or rejects a particle with a size between about 700 microns and about 1000 microns. In some embodiments, the filter may comprise a pore that retains or rejects a particle with a size between about 700 microns and about 900 microns. In some embodiments, the filter may comprise a pore that retains or rejects a particle with a size between about 700 microns and about 800 microns. In some embodiments, the filter may comprise a pore that retains or rejects a particle with a size between about 800 microns and about 1000 microns. In some embodiments, the filter may comprise a pore that retains or rejects a particle with a size between about 800 microns and about 900 microns. In some embodiments, the filter may comprise a pore that retains or rejects a particle with a size between about 900 microns and about 1000 microns.

In some embodiments, the filter may comprise a pore that retains or rejects a particle with a size between about 1 micron and about 100 microns, e.g., about 1 micron, 2 microns, 3 microns, 4 microns, 5 microns, 6 microns, 7 microns, 8 microns, 9 microns, 10 microns, 11 microns, 12 microns, 13 microns, 14 microns, 15 microns, 16 microns, 17 microns, 18 microns, 19 microns, 20 microns, 21 microns, 22 microns, 23 microns, 24 microns, 25 microns, 26 microns, 27 microns, 28 microns, 29 microns, 30 microns, 31 microns, 32 microns, 33 microns, 34 microns, 35 microns, 36 microns, 37 microns, 38 microns, 39 microns, 40 microns, 41 microns, 42 microns, 43 microns, 44 microns, 45 microns, 46 microns, 47 microns, 48 microns, 49 microns, 50 microns, 51 microns, 52 microns, 53 microns, 54 microns, 55 microns, 56 microns, 57 microns, 58 microns, 59 microns, 60 microns, 61 microns, 62 microns, 63 microns, 64 microns, 65 microns, 66 microns, 67 microns, 68 microns, 69 microns, 70 microns, 71 microns, 72 microns, 73 microns, 74 microns, 75 microns, 76 microns, 77 microns, 78 microns, 79 microns, 80 microns, 81 microns, 82 microns, 83 microns, 84 microns, 85 microns, 86 microns, 87 microns, 88 microns, 89 microns, 90 microns, 91 microns, 92 microns, 93 microns, 94 microns, 95 microns, 96 microns, 97 microns, 98 microns, 99 microns, or about 100 microns, or any size therebetween.

In some embodiments, the filter may comprise a pore that retains or rejects a particle with a size between about 10 microns and about 100 microns. In some embodiments, the filter may comprise a pore that retains or rejects a particle with a size between about 10 microns and about 90 microns. In some embodiments, the filter may comprise a pore that retains or rejects a particle with a size between about 10 microns and about 80 microns. In some embodiments, the filter may comprise a pore that retains or rejects a particle with a size between about 10 microns and about 70 microns. In some embodiments, the filter may comprise a pore that retains or rejects a particle with a size between about 10 microns and about 60 microns. In some embodiments, the filter may comprise a pore that retains or rejects a particle with a size between about 10 microns and about 50 microns. In some embodiments, the filter may comprise a pore that retains or rejects a particle with a size between about 10 microns and about 40 microns. In some embodiments, the filter may comprise a pore that retains or rejects a particle with a size between about 10 microns and about 30 microns. In some embodiments, the filter may comprise a pore that retains or rejects a particle with a size between about 10 microns and about 20 microns. In some embodiments, the filter may comprise a pore that retains or rejects a particle with a size between about 20 microns and about 100 microns. In some embodiments, the filter may comprise a pore that retains or rejects a particle with a size between about 20 microns and about 90 microns. In some embodiments, the filter may comprise a pore that retains or rejects a particle with a size between about 20 microns and about 80 microns. In some embodiments, the filter may comprise a pore that retains or rejects a particle with a size between about 20 microns and about 70 microns. In some embodiments, the filter may comprise a pore that retains or rejects a particle with a size between about 20 microns and about 60 microns. In some embodiments, the filter may comprise a pore that retains or rejects a particle with a size between about 20 microns and about 50 microns. In some embodiments, the filter may comprise a pore that retains or rejects a particle with a size between about 20 microns and about 40 microns. In some embodiments, the filter may comprise a pore that retains or rejects a particle with a size between about 20 microns and about 30 microns. In some embodiments, the filter may comprise a pore that retains or rejects a particle with a size between about 30 microns and about 100 microns. In some embodiments, the filter may comprise a pore that retains or rejects a particle with a size between about 30 microns and about 90 microns. In some embodiments, the filter may comprise a pore that retains or rejects a particle with a size between about 30 microns and about 80 microns. In some embodiments, the filter may comprise a pore that retains or rejects a particle with a size between about 30 microns and about 70 microns. In some embodiments, the filter may comprise a pore that retains or rejects a particle with a size between about 30 microns and about 60 microns. In some embodiments, the filter may comprise a pore that retains or rejects a particle with a size between about 30 microns and about 50 microns. In some embodiments, the filter may comprise a pore that retains or rejects a particle with a size between about 30 microns and about 40 microns. In some embodiments, the filter may comprise a pore that retains or rejects a particle with a size between about 40 microns and about 100 microns. In some embodiments, the filter may comprise a pore that retains or rejects a particle with a size between about 40 microns and about 90 microns. In some embodiments, the filter may comprise a pore that retains or rejects a particle with a size between about 40 microns and about 80 microns. In some embodiments, the filter may comprise a pore that retains or rejects a particle with a size between about 40 microns and about 70 microns. In some embodiments, the filter may comprise a pore that retains or rejects a particle with a size between about 40 microns and about 60 microns. In some embodiments, the filter may comprise a pore that retains or rejects a particle with a size between about 40 microns and about 50 microns. In some embodiments, the filter may comprise a pore that retains or rejects a particle with a size between about 50 microns and about 100 microns. In some embodiments, the filter may comprise a pore that retains or rejects a particle with a size between about 50 microns and about 90 microns. In some embodiments, the filter may comprise a pore that retains or rejects a particle with a size between about 50 microns and about 80 microns. In some embodiments, the filter may comprise a pore that retains or rejects a particle with a size between about 50 microns and about 70 microns. In some embodiments, the filter may comprise a pore that retains or rejects a particle with a size between about 50 microns and about 60 microns. In some embodiments, the filter may comprise a pore that retains or rejects a particle with a size between about 60 microns and about 100 microns. In some embodiments, the filter may comprise a pore that retains or rejects a particle with a size between about 60 microns and about 90 microns. In some embodiments, the filter may comprise a pore that retains or rejects a particle with a size between about 60 microns and about 80 microns. In some embodiments, the filter may comprise a pore that retains or rejects a particle with a size between about 60 microns and about 70 microns. In some embodiments, the filter may comprise a pore that retains or rejects a particle with a size between about 60 microns and about 100 microns. In some embodiments, the filter may comprise a pore that retains or rejects a particle with a size between about 70 microns and about 100 microns. In some embodiments, the filter may comprise a pore that retains or rejects a particle with a size between about 70 microns and about 90 microns. In some embodiments, the filter may comprise a pore that retains or rejects a particle with a size between about 70 microns and about 80 microns. In some embodiments, the filter may comprise a pore that retains or rejects a particle with a size between about 80 microns and about 100 microns. In some embodiments, the filter may comprise a pore that retains or rejects a particle with a size between about 80 microns and about 90 microns. In some embodiments, the filter may comprise a pore that retains or rejects a particle with a size between about 90 microns and about 100 microns.

In some embodiments, the filter may comprise a pore that retains or rejects a particle with a size between about 1 micron and about 10 microns. In some embodiments, the filter may comprise a pore that retains or rejects a particle with a size between about 1 micron and about 9 microns. In some embodiments, the filter may comprise a pore that retains or rejects a particle with a size between about 1 micron and about 8 microns. In some embodiments, the filter may comprise a pore that retains or rejects a particle with a size between about 1 micron and about 7 microns. In some embodiments, the filter may comprise a pore that retains or rejects a particle with a size between about 1 micron and about 6 microns. In some embodiments, the filter may comprise a pore that retains or rejects a particle with a size between about 1 micron and about 5 microns. In some embodiments, the filter may comprise a pore that retains or rejects a particle with a size between about 1 micron and about 4 microns. In some embodiments, the filter may comprise a pore that retains or rejects a particle with a size between about 1 micron and about 3 microns. In some embodiments, the filter may comprise a pore that retains or rejects a particle with a size between about 1 micron and about 2 microns. In some embodiments, the filter may comprise a pore that retains or rejects a particle with a size between about 2 microns and about 10 microns. In some embodiments, the filter may comprise a pore that retains or rejects a particle with a size between about 2 microns and about 9 microns. In some embodiments, the filter may comprise a pore that retains or rejects a particle with a size between about 2 microns and about 8 microns. In some embodiments, the filter may comprise a pore that retains or rejects a particle with a size between about 2 microns and about 7 microns. In some embodiments, the filter may comprise a pore that retains or rejects a particle with a size between about 2 microns and about 6 microns. In some embodiments, the filter may comprise a pore that retains or rejects a particle with a size between about 2 microns and about 5 microns. In some embodiments, the filter may comprise a pore that retains or rejects a particle with a size between about 2 microns and about 4 microns. In some embodiments, the filter may comprise a pore that retains or rejects a particle with a size between about 2 microns and about 3 microns. In some embodiments, the filter may comprise a pore that retains or rejects a particle with a size between about 3 microns and about 10 microns. In some embodiments, the filter may comprise a pore that retains or rejects a particle with a size between about 3 microns and about 9 microns. In some embodiments, the filter may comprise a pore that retains or rejects a particle with a size between about 3 microns and about 8 microns. In some embodiments, the filter may comprise a pore that retains or rejects a particle with a size between about 3 microns and about 7 microns. In some embodiments, the filter may comprise a pore that retains or rejects a particle with a size between about 3 microns and about 6 microns. In some embodiments, the filter may comprise a pore that retains or rejects a particle with a size between about 3 microns and about 5 microns. In some embodiments, the filter may comprise a pore that retains or rejects a particle with a size between about 3 microns and about 4 microns. In some embodiments, the filter may comprise a pore that retains or rejects a particle with a size between about 4 microns and about 10 microns. In some embodiments, the filter may comprise a pore that retains or rejects a particle with a size between about 4 microns and about 9 microns. In some embodiments, the filter may comprise a pore that retains or rejects a particle with a size between about 4 microns and about 8 microns. In some embodiments, the filter may comprise a pore that retains or rejects a particle with a size between about 4 microns and about 7 microns. In some embodiments, the filter may comprise a pore that retains or rejects a particle with a size between about 4 microns and about 6 microns. In some embodiments, the filter may comprise a pore that retains or rejects a particle with a size between about 4 microns and about 5 microns. In some embodiments, the filter may comprise a pore that retains or rejects a particle with a size between about 5 microns and about 10 microns. In some embodiments, the filter may comprise a pore that retains or rejects a particle with a size between about 5 microns and about 9 microns. In some embodiments, the filter may comprise a pore that retains or rejects a particle with a size between about 5 microns and about 8 microns. In some embodiments, the filter may comprise a pore that retains or rejects a particle with a size between about 5 microns and about 7 microns. In some embodiments, the filter may comprise a pore that retains or rejects a particle with a size between about 5 microns and about 6 microns. In some embodiments, the filter may comprise a pore that retains or rejects a particle with a size between about 6 microns and about 10 microns. In some embodiments, the filter may comprise a pore that retains or rejects a particle with a size between about 6 microns and about 9 microns. In some embodiments, the filter may comprise a pore that retains or rejects a particle with a size between about 6 microns and about 8 microns. In some embodiments, the filter may comprise a pore that retains or rejects a particle with a size between about 6 microns and about 7 microns. In some embodiments, the filter may comprise a pore that retains or rejects a particle with a size between about 6 microns and about 10 microns. In some embodiments, the filter may comprise a pore that retains or rejects a particle with a size between about 7 microns and about 10 microns. In some embodiments, the filter may comprise a pore that retains or rejects a particle with a size between about 7 microns and about 9 microns. In some embodiments, the filter may comprise a pore that retains or rejects a particle with a size between about 7 microns and about 8 microns. In some embodiments, the filter may comprise a pore that retains or rejects a particle with a size between about 8 microns and about 10 microns. In some embodiments, the filter may comprise a pore that retains or rejects a particle with a size between about 8 microns and about 9 microns. In some embodiments, the filter may comprise a pore that retains or rejects a particle with a size between about 9 microns and about 10 microns.

In some embodiments, the filter may comprise a pore that retains or rejects a particle with a size between about 0.001 micron and about 1.0 micron, e.g., about 0.001 micron, 0.002 micron, 0.003 micron, 0.004 micron, 0.005 micron, 0.006 micron, 0.007 micron, 0.008 micron, 0.009 micron, 0.01 micron, 0.02 micron, 0.03 micron, 0.04 micron, 0.05 micron, 0.06 micron, 0.07 micron, 0.08 micron, 0.09 micron, 0.1 micron, 0.11 micron, 0.12 micron, 0.13 micron, 0.14 micron, 0.15 micron, 0.16 micron, 0.17 micron, 0.18 micron, 0.19 micron, 0.2 micron, 0.21 micron, 0.22 micron, 0.23 micron, 0.24 micron, 0.25 micron, 0.26 micron, 0.27 micron, 0.28 micron, 0.29 micron, 0.3 micron, 0.31 micron, 0.32 micron, 0.33 micron, 0.34 micron, 0.35 micron, 0.36 micron, 0.37 micron, 0.38 micron, 0.39 micron, 0.4 micron, 0.41 micron, 0.42 micron, 0.43 micron, 0.44 micron, 0.45 micron, 0.46 micron, 0.47 micron, 0.48 micron, 0.49 micron, 0.5 micron, 0.51 micron, 0.52 micron, 0.53 micron, 0.54 micron, 0.55 micron, 0.56 micron, 0.57 micron, 0.58 micron, 0.59 micron, 0.6 micron, 0.61 micron, 0.62 micron, 0.63 micron, 0.64 micron, 0.65 micron, 0.66 micron, 0.67 micron, 0.68 micron, 0.69 micron, 0.7 micron, 0.71 micron, 0.72 micron, 0.73 micron, 0.74 micron, 0.75 micron, 0.76 micron, 0.77 micron, 0.78 micron, 0.79 micron, 0.8 micron, 0.81 micron, 0.82 micron, 0.83 micron, 0.84 micron, 0.85 micron, 0.86 micron, 0.87 micron, 0.88 micron, 0.89 micron, 0.9 micron, 0.91 micron, 0.92 micron, 0.93 micron, 0.94 micron, 0.95 micron, 0.96 micron, 0.97 micron, 0.98 micron, 0.99 micron, or about 1.0 micron, or any size therebetween.

In some embodiments, the filter may comprise a pore that retains or rejects a particle with a size between about 0.1 micron and about 1.0 micron. In some embodiments, the filter may comprise a pore that retains or rejects a particle with a size between about 0.1 micron and about 0.9 micron. In some embodiments, the filter may comprise a pore that retains or rejects a particle with a size between about 0.1 micron and about 0.8 micron. In some embodiments, the filter may comprise a pore that retains or rejects a particle with a size between about 0.1 micron and about 0.7 micron. In some embodiments, the filter may comprise a pore that retains or rejects a particle with a size between about 0.1 micron and about 0.6 micron. In some embodiments, the filter may comprise a pore that retains or rejects a particle with a size between about 0.1 micron and about 0.5 micron. In some embodiments, the filter may comprise a pore that retains or rejects a particle with a size between about 0.1 micron and about 0.4 micron. In some embodiments, the filter may comprise a pore that retains or rejects a particle with a size between about 0.1 micron and about 0.3 micron. In some embodiments, the filter may comprise a pore that retains or rejects a particle with a size between about 0.1 micron and about 0.2 micron. In some embodiments, the filter may comprise a pore that retains or rejects a particle with a size between about 0.2 micron and about 1.0 micron. In some embodiments, the filter may comprise a pore that retains or rejects a particle with a size between about 0.2 micron and about 0.9 micron. In some embodiments, the filter may comprise a pore that retains or rejects a particle with a size between about 0.2 micron and about 0.8 micron. In some embodiments, the filter may comprise a pore that retains or rejects a particle with a size between about 0.2 micron and about 0.7 micron. In some embodiments, the filter may comprise a pore that retains or rejects a particle with a size between about 0.2 micron and about 0.6 micron. In some embodiments, the filter may comprise a pore that retains or rejects a particle with a size between about 0.2 micron and about 0.5 micron. In some embodiments, the filter may comprise a pore that retains or rejects a particle with a size between about 0.2 micron and about 0.4 micron. In some embodiments, the filter may comprise a pore that retains or rejects a particle with a size between about 0.2 micron and about 0.3 micron. In some embodiments, the filter may comprise a pore that retains or rejects a particle with a size between about 0.3 micron and about 1.0 micron. In some embodiments, the filter may comprise a pore that retains or rejects a particle with a size between about 0.3 micron and about 0.9 micron. In some embodiments, the filter may comprise a pore that retains or rejects a particle with a size between about 0.3 micron and about 0.8 micron. In some embodiments, the filter may comprise a pore that retains or rejects a particle with a size between about 0.3 micron and about 0.7 micron. In some embodiments, the filter may comprise a pore that retains or rejects a particle with a size between about 0.3 micron and about 0.6 micron. In some embodiments, the filter may comprise a pore that retains or rejects a particle with a size between about 0.3 micron and about 0.5 micron. In some embodiments, the filter may comprise a pore that retains or rejects a particle with a size between about 0.3 micron and about 0.4 micron. In some embodiments, the filter may comprise a pore that retains or rejects a particle with a size between about 0.4 micron and about 1.0 micron. In some embodiments, the filter may comprise a pore that retains or rejects a particle with a size between about 0.4 micron and about 0.9 micron. In some embodiments, the filter may comprise a pore that retains or rejects a particle with a size between about 0.4 micron and about 0.8 micron. In some embodiments, the filter may comprise a pore that retains or rejects a particle with a size between about 0.4 micron and about 0.7 micron. In some embodiments, the filter may comprise a pore that retains or rejects a particle with a size between about 0.4 micron and about 0.6 micron. In some embodiments, the filter may comprise a pore that retains or rejects a particle with a size between about 0.4 micron and about 0.5 micron. In some embodiments, the filter may comprise a pore that retains or rejects a particle with a size between about 0.5 micron and about 1.0 micron. In some embodiments, the filter may comprise a pore that retains or rejects a particle with a size between about 0.5 micron and about 0.9 micron. In some embodiments, the filter may comprise a pore that retains or rejects a particle with a size between about 0.5 micron and about 0.8 micron. In some embodiments, the filter may comprise a pore that retains or rejects a particle with a size between about 0.5 micron and about 0.7 micron. In some embodiments, the filter may comprise a pore that retains or rejects a particle with a size between about 0.5 micron and about 0.6 micron. In some embodiments, the filter may comprise a pore that retains or rejects a particle with a size between about 0.6 micron and about 1.0 micron. In some embodiments, the filter may comprise a pore that retains or rejects a particle with a size between about 0.6 micron and about 0.9 micron. In some embodiments, the filter may comprise a pore that retains or rejects a particle with a size between about 0.6 micron and about 0.8 micron. In some embodiments, the filter may comprise a pore that retains or rejects a particle with a size between about 0.6 micron and about 0.7 micron. In some embodiments, the filter may comprise a pore that retains or rejects a particle with a size between about 0.7 micron and about 1.0 micron. In some embodiments, the filter may comprise a pore that retains or rejects a particle with a size between about 0.7 micron and about 0.9 micron. In some embodiments, the filter may comprise a pore that retains or rejects a particle with a size between about 0.7 micron and about 0.8 micron. In some embodiments, the filter may comprise a pore that retains or rejects a particle with a size between about 0.8 micron and about 1.0 micron. In some embodiments, the filter may comprise a pore that retains or rejects a particle with a size between about 0.8 micron and about 0.9 micron. In some embodiments, the filter may comprise a pore that retains or rejects a particle with a size between about 0.9 micron and about 1.0 micron.

In some embodiments, the filter may comprise a pore that retains or rejects a particle with a size between about 0.01 micron and about 0.1 micron. In some embodiments, the filter may comprise a pore that retains or rejects a particle with a size between about 0.01 micron and about 0.09 micron. In some embodiments, the filter may comprise a pore that retains or rejects a particle with a size between about 0.01 micron and about 0.08 micron. In some embodiments, the filter may comprise a pore that retains or rejects a particle with a size between about 0.01 micron and about 0.07 micron. In some embodiments, the filter may comprise a pore that retains or rejects a particle with a size between about 0.01 micron and about 0.06 micron. In some embodiments, the filter may comprise a pore that retains or rejects a particle with a size between about 0.01 micron and about 0.05 micron. In some embodiments, the filter may comprise a pore that retains or rejects a particle with a size between about 0.01 micron and about 0.04 micron. In some embodiments, the filter may comprise a pore that retains or rejects a particle with a size between about 0.01 micron and about 0.03 micron. In some embodiments, the filter may comprise a pore that retains or rejects a particle with a size between about 0.01 micron and about 0.02 micron. In some embodiments, the filter may comprise a pore that retains or rejects a particle with a size between about 0.02 micron and about 0.1 micron. In some embodiments, the filter may comprise a pore that retains or rejects a particle with a size between about 0.02 micron and about 0.09 micron. In some embodiments, the filter may comprise a pore that retains or rejects a particle with a size between about 0.02 micron and about 0.08 micron. In some embodiments, the filter may comprise a pore that retains or rejects a particle with a size between about 0.02 micron and about 0.07 micron. In some embodiments, the filter may comprise a pore that retains or rejects a particle with a size between about 0.02 micron and about 0.06 micron. In some embodiments, the filter may comprise a pore that retains or rejects a particle with a size between about 0.02 micron and about 0.05 micron. In some embodiments, the filter may comprise a pore that retains or rejects a particle with a size between about 0.02 micron and about 0.04 micron. In some embodiments, the filter may comprise a pore that retains or rejects a particle with a size between about 0.02 micron and about 0.03 micron. In some embodiments, the filter may comprise a pore that retains or rejects a particle with a size between about 0.03 micron and about 0.1 micron. In some embodiments, the filter may comprise a pore that retains or rejects a particle with a size between about 0.03 micron and about 0.09 micron. In some embodiments, the filter may comprise a pore that retains or rejects a particle with a size between about 0.03 micron and about 0.08 micron. In some embodiments, the filter may comprise a pore that retains or rejects a particle with a size between about 0.03 micron and about 0.07 micron. In some embodiments, the filter may comprise a pore that retains or rejects a particle with a size between about 0.03 micron and about 0.06 micron. In some embodiments, the filter may comprise a pore that retains or rejects a particle with a size between about 0.03 micron and about 0.05 micron. In some embodiments, the filter may comprise a pore that retains or rejects a particle with a size between about 0.03 micron and about 0.04 micron. In some embodiments, the filter may comprise a pore that retains or rejects a particle with a size between about 0.04 micron and about 0.1 micron. In some embodiments, the filter may comprise a pore that retains or rejects a particle with a size between about 0.04 micron and about 0.09 micron. In some embodiments, the filter may comprise a pore that retains or rejects a particle with a size between about 0.04 micron and about 0.08 micron. In some embodiments, the filter may comprise a pore that retains or rejects a particle with a size between about 0.04 micron and about 0.07 micron. In some embodiments, the filter may comprise a pore that retains or rejects a particle with a size between about 0.04 micron and about 0.06 micron. In some embodiments, the filter may comprise a pore that retains or rejects a particle with a size between about 0.04 micron and about 0.05 micron. In some embodiments, the filter may comprise a pore that retains or rejects a particle with a size between about 0.05 micron and about 0.1 micron. In some embodiments, the filter may comprise a pore that retains or rejects a particle with a size between about 0.05 micron and about 0.09 micron. In some embodiments, the filter may comprise a pore that retains or rejects a particle with a size between about 0.05 micron and about 0.08 micron. In some embodiments, the filter may comprise a pore that retains or rejects a particle with a size between about 0.05 micron and about 0.07 micron. In some embodiments, the filter may comprise a pore that retains or rejects a particle with a size between about 0.05 micron and about 0.06 micron. In some embodiments, the filter may comprise a pore that retains or rejects a particle with a size between about 0.06 micron and about 0.1 micron. In some embodiments, the filter may comprise a pore that retains or rejects a particle with a size between about 0.06 micron and about 0.09 micron. In some embodiments, the filter may comprise a pore that retains or rejects a particle with a size between about 0.06 micron and about 0.08 micron. In some embodiments, the filter may comprise a pore that retains or rejects a particle with a size between about 0.06 micron and about 0.07 micron. In some embodiments, the filter may comprise a pore that retains or rejects a particle with a size between about 0.07 micron and about 0.1 micron. In some embodiments, the filter may comprise a pore that retains or rejects a particle with a size between about 0.07 micron and about 0.09 micron. In some embodiments, the filter may comprise a pore that retains or rejects a particle with a size between about 0.07 micron and about 0.08 micron. In some embodiments, the filter may comprise a pore that retains or rejects a particle with a size between about 0.08 micron and about 0.1 micron. In some embodiments, the filter may comprise a pore that retains or rejects a particle with a size between about 0.08 micron and about 0.09 micron. In some embodiments, the filter may comprise a pore that retains or rejects a particle with a size between about 0.09 micron and about 0.1 micron.

In some embodiments, the filter may comprise a pore that retains or rejects a particle with a size between about 0.001 micron and about 0.01 micron. In some embodiments, the filter may comprise a pore that retains or rejects a particle with a size between about 0.001 micron and about 0.009 micron. In some embodiments, the filter may comprise a pore that retains or rejects a particle with a size between about 0.001 micron and about 0.008 micron. In some embodiments, the filter may comprise a pore that retains or rejects a particle with a size between about 0.001 micron and about 0.007 micron. In some embodiments, the filter may comprise a pore that retains or rejects a particle with a size between about 0.001 micron and about 0.006 micron. In some embodiments, the filter may comprise a pore that retains or rejects a particle with a size between about 0.001 micron and about 0.005 micron. In some embodiments, the filter may comprise a pore that retains or rejects a particle with a size between about 0.001 micron and about 0.004 micron. In some embodiments, the filter may comprise a pore that retains or rejects a particle with a size between about 0.001 micron and about 0.003 micron. In some embodiments, the filter may comprise a pore that retains or rejects a particle with a size between about 0.001 micron and about 0.002 micron. In some embodiments, the filter may comprise a pore that retains or rejects a particle with a size between about 0.002 micron and about 0.01 micron. In some embodiments, the filter may comprise a pore that retains or rejects a particle with a size between about 0.002 micron and about 0.009 micron. In some embodiments, the filter may comprise a pore that retains or rejects a particle with a size between about 0.002 micron and about 0.008 micron. In some embodiments, the filter may comprise a pore that retains or rejects a particle with a size between about 0.002 micron and about 0.007 micron. In some embodiments, the filter may comprise a pore that retains or rejects a particle with a size between about 0.002 micron and about 0.006 micron. In some embodiments, the filter may comprise a pore that retains or rejects a particle with a size between about 0.002 micron and about 0.005 micron. In some embodiments, the filter may comprise a pore that retains or rejects a particle with a size between about 0.002 micron and about 0.004 micron. In some embodiments, the filter may comprise a pore that retains or rejects a particle with a size between about 0.002 micron and about 0.003 micron. In some embodiments, the filter may comprise a pore that retains or rejects a particle with a size between about 0.003 micron and about 0.01 micron. In some embodiments, the filter may comprise a pore that retains or rejects a particle with a size between about 0.003 micron and about 0.009 micron. In some embodiments, the filter may comprise a pore that retains or rejects a particle with a size between about 0.003 micron and about 0.008 micron. In some embodiments, the filter may comprise a pore that retains or rejects a particle with a size between about 0.003 micron and about 0.007 micron. In some embodiments, the filter may comprise a pore that retains or rejects a particle with a size between about 0.003 micron and about 0.006 micron. In some embodiments, the filter may comprise a pore that retains or rejects a particle with a size between about 0.003 micron and about 0.005 micron. In some embodiments, the filter may comprise a pore that retains or rejects a particle with a size between about 0.003 micron and about 0.004 micron. In some embodiments, the filter may comprise a pore that retains or rejects a particle with a size between about 0.004 micron and about 0.01 micron. In some embodiments, the filter may comprise a pore that retains or rejects a particle with a size between about 0.004 micron and about 0.009 micron. In some embodiments, the filter may comprise a pore that retains or rejects a particle with a size between about 0.004 micron and about 0.008 micron. In some embodiments, the filter may comprise a pore that retains or rejects a particle with a size between about 0.004 micron and about 0.007 micron. In some embodiments, the filter may comprise a pore that retains or rejects a particle with a size between about 0.004 micron and about 0.006 micron. In some embodiments, the filter may comprise a pore that retains or rejects a particle with a size between about 0.004 micron and about 0.005 micron. In some embodiments, the filter may comprise a pore that retains or rejects a particle with a size between about 0.005 micron and about 0.01 micron. In some embodiments, the filter may comprise a pore that retains or rejects a particle with a size between about 0.005 micron and about 0.009 micron. In some embodiments, the filter may comprise a pore that retains or rejects a particle with a size between about 0.005 micron and about 0.008 micron. In some embodiments, the filter may comprise a pore that retains or rejects a particle with a size between about 0.005 micron and about 0.007 micron. In some embodiments, the filter may comprise a pore that retains or rejects a particle with a size between about 0.005 micron and about 0.006 micron. In some embodiments, the filter may comprise a pore that retains or rejects a particle with a size between about 0.006 micron and about 0.01 micron. In some embodiments, the filter may comprise a pore that retains or rejects a particle with a size between about 0.006 micron and about 0.009 micron. In some embodiments, the filter may comprise a pore that retains or rejects a particle with a size between about 0.006 micron and about 0.008 micron. In some embodiments, the filter may comprise a pore that retains or rejects a particle with a size between about 0.006 micron and about 0.007 micron. In some embodiments, the filter may comprise a pore that retains or rejects a particle with a size between about 0.007 micron and about 0.01 micron. In some embodiments, the filter may comprise a pore that retains or rejects a particle with a size between about 0.007 micron and about 0.009 micron. In some embodiments, the filter may comprise a pore that retains or rejects a particle with a size between about 0.007 micron and about 0.008 micron. In some embodiments, the filter may comprise a pore that retains or rejects a particle with a size between about 0.008 micron and about 0.01 micron. In some embodiments, the filter may comprise a pore that retains or rejects a particle with a size between about 0.008 micron and about 0.009 micron. In some embodiments, the filter may comprise a pore that retains or rejects a particle with a size between about 0.009 micron and about 0.01 micron.

In some embodiments, the filter may comprise a pore that retains or rejects a particle with a size between about 0.1 micron and about 100 microns. In some embodiments, the filter may comprise a pore that retains or rejects a particle with a size between about 0.1 micron and about 95 microns. In some embodiments, the filter may comprise a pore that retains or rejects a particle with a size between about 0.1 micron and about 90 microns. In some embodiments, the filter may comprise a pore that retains or rejects a particle with a size between about 0.1 micron and about 85 microns. In some embodiments, the filter may comprise a pore that retains or rejects a particle with a size between about 0.1 micron and about 80 microns. In some embodiments, the filter may comprise a pore that retains or rejects a particle with a size between about 0.1 micron and about 75 microns. In some embodiments, the filter may comprise a pore that retains or rejects a particle with a size between about 0.1 micron and about 70 microns. In some embodiments, the filter may comprise a pore that retains or rejects a particle with a size between about 0.1 micron and about 65 microns. In some embodiments, the filter may comprise a pore that retains or rejects a particle with a size between about 0.1 micron and about 60 microns. In some embodiments, the filter may comprise a pore that retains or rejects a particle with a size between about 0.1 micron and about 55 microns. In some embodiments, the filter may comprise a pore that retains or rejects a particle with a size between about 0.1 micron and about 50 microns. In some embodiments, the filter may comprise a pore that retains or rejects a particle with a size between about 0.1 micron and about 45 microns. In some embodiments, the filter may comprise a pore that retains or rejects a particle with a size between about 0.1 micron and about 40 microns. In some embodiments, the filter may comprise a pore that retains or rejects a particle with a size between about 0.1 micron and about 35 microns. In some embodiments, the filter may comprise a pore that retains or rejects a particle with a size between about 0.1 micron and about 30 microns. In some embodiments, the filter may comprise a pore that retains or rejects a particle with a size between about 0.1 micron and about 25 microns. In some embodiments, the filter may comprise a pore that retains or rejects a particle with a size between about 0.1 micron and about 20 microns. In some embodiments, the filter may comprise a pore that retains or rejects a particle with a size between about 0.1 micron and about 15 microns. In some embodiments, the filter may comprise a pore that retains or rejects a particle with a size between about 0.1 micron and about 10 microns. In some embodiments, the filter may comprise a pore that retains or rejects a particle with a size between about 0.1 micron and about 5 microns.

In some embodiments, the filter may comprise a pore that retains or rejects a particle with a size between about 0.2 micron and about 100 microns. In some embodiments, the filter may comprise a pore that retains or rejects a particle with a size between about 0.2 micron and about 95 microns. In some embodiments, the filter may comprise a pore that retains or rejects a particle with a size between about 0.2 micron and about 90 microns. In some embodiments, the filter may comprise a pore that retains or rejects a particle with a size between about 0.2 micron and about 85 microns. In some embodiments, the filter may comprise a pore that retains or rejects a particle with a size between about 0.2 micron and about 80 microns. In some embodiments, the filter may comprise a pore that retains or rejects a particle with a size between about 0.2 micron and about 75 microns. In some embodiments, the filter may comprise a pore that retains or rejects a particle with a size between about 0.2 micron and about 70 microns. In some embodiments, the filter may comprise a pore that retains or rejects a particle with a size between about 0.2 micron and about 65 microns. In some embodiments, the filter may comprise a pore that retains or rejects a particle with a size between about 0.2 micron and about 60 microns. In some embodiments, the filter may comprise a pore that retains or rejects a particle with a size between about 0.2 micron and about 55 microns. In some embodiments, the filter may comprise a pore that retains or rejects a particle with a size between about 0.2 micron and about 50 microns. In some embodiments, the filter may comprise a pore that retains or rejects a particle with a size between about 0.2 micron and about 45 microns. In some embodiments, the filter may comprise a pore that retains or rejects a particle with a size between about 0.2 micron and about 40 microns. In some embodiments, the filter may comprise a pore that retains or rejects a particle with a size between about 0.2 micron and about 35 microns. In some embodiments, the filter may comprise a pore that retains or rejects a particle with a size between about 0.2 micron and about 30 microns. In some embodiments, the filter may comprise a pore that retains or rejects a particle with a size between about 0.2 micron and about 25 microns. In some embodiments, the filter may comprise a pore that retains or rejects a particle with a size between about 0.2 micron and about 20 microns. In some embodiments, the filter may comprise a pore that retains or rejects a particle with a size between about 0.2 micron and about 15 microns. In some embodiments, the filter may comprise a pore that retains or rejects a particle with a size between about 0.2 micron and about 10 microns. In some embodiments, the filter may comprise a pore that retains or rejects a particle with a size between about 0.2 micron and about 5 microns.

In some embodiments, the filter may comprise a pore that retains or rejects a particle with a size between about 0.3 micron and about 100 microns. In some embodiments, the filter may comprise a pore that retains or rejects a particle with a size between about 0.3 micron and about 95 microns. In some embodiments, the filter may comprise a pore that retains or rejects a particle with a size between about 0.3 micron and about 90 microns. In some embodiments, the filter may comprise a pore that retains or rejects a particle with a size between about 0.3 micron and about 85 microns. In some embodiments, the filter may comprise a pore that retains or rejects a particle with a size between about 0.3 micron and about 80 microns. In some embodiments, the filter may comprise a pore that retains or rejects a particle with a size between about 0.3 micron and about 75 microns. In some embodiments, the filter may comprise a pore that retains or rejects a particle with a size between about 0.3 micron and about 70 microns. In some embodiments, the filter may comprise a pore that retains or rejects a particle with a size between about 0.3 micron and about 65 microns. In some embodiments, the filter may comprise a pore that retains or rejects a particle with a size between about 0.3 micron and about 60 microns. In some embodiments, the filter may comprise a pore that retains or rejects a particle with a size between about 0.3 micron and about 55 microns. In some embodiments, the filter may comprise a pore that retains or rejects a particle with a size between about 0.3 micron and about 50 microns. In some embodiments, the filter may comprise a pore that retains or rejects a particle with a size between about 0.3 micron and about 45 microns. In some embodiments, the filter may comprise a pore that retains or rejects a particle with a size between about 0.3 micron and about 40 microns. In some embodiments, the filter may comprise a pore that retains or rejects a particle with a size between about 0.3 micron and about 35 microns. In some embodiments, the filter may comprise a pore that retains or rejects a particle with a size between about 0.3 micron and about 30 microns. In some embodiments, the filter may comprise a pore that retains or rejects a particle with a size between about 0.3 micron and about 25 microns. In some embodiments, the filter may comprise a pore that retains or rejects a particle with a size between about 0.3 micron and about 20 microns. In some embodiments, the filter may comprise a pore that retains or rejects a particle with a size between about 0.3 micron and about 15 microns. In some embodiments, the filter may comprise a pore that retains or rejects a particle with a size between about 0.3 micron and about 10 microns. In some embodiments, the filter may comprise a pore that retains or rejects a particle with a size between about 0.3 micron and about 5 microns.

In some embodiments, the filter may comprise a pore that retains or rejects a particle with a size between about 0.4 micron and about 100 microns. In some embodiments, the filter may comprise a pore that retains or rejects a particle with a size between about 0.4 micron and about 95 microns. In some embodiments, the filter may comprise a pore that retains or rejects a particle with a size between about 0.4 micron and about 90 microns. In some embodiments, the filter may comprise a pore that retains or rejects a particle with a size between about 0.4 micron and about 85 microns. In some embodiments, the filter may comprise a pore that retains or rejects a particle with a size between about 0.4 micron and about 80 microns. In some embodiments, the filter may comprise a pore that retains or rejects a particle with a size between about 0.4 micron and about 75 microns. In some embodiments, the filter may comprise a pore that retains or rejects a particle with a size between about 0.4 micron and about 70 microns. In some embodiments, the filter may comprise a pore that retains or rejects a particle with a size between about 0.4 micron and about 65 microns. In some embodiments, the filter may comprise a pore that retains or rejects a particle with a size between about 0.4 micron and about 60 microns. In some embodiments, the filter may comprise a pore that retains or rejects a particle with a size between about 0.4 micron and about 55 microns. In some embodiments, the filter may comprise a pore that retains or rejects a particle with a size between about 0.4 micron and about 50 microns. In some embodiments, the filter may comprise a pore that retains or rejects a particle with a size between about 0.4 micron and about 45 microns. In some embodiments, the filter may comprise a pore that retains or rejects a particle with a size between about 0.4 micron and about 40 microns. In some embodiments, the filter may comprise a pore that retains or rejects a particle with a size between about 0.4 micron and about 35 microns. In some embodiments, the filter may comprise a pore that retains or rejects a particle with a size between about 0.4 micron and about 30 microns. In some embodiments, the filter may comprise a pore that retains or rejects a particle with a size between about 0.4 micron and about 25 microns. In some embodiments, the filter may comprise a pore that retains or rejects a particle with a size between about 0.4 micron and about 20 microns. In some embodiments, the filter may comprise a pore that retains or rejects a particle with a size between about 0.4 micron and about 15 microns. In some embodiments, the filter may comprise a pore that retains or rejects a particle with a size between about 0.4 micron and about 10 microns. In some embodiments, the filter may comprise a pore that retains or rejects a particle with a size between about 0.4 micron and about 5 microns.

In some embodiments, the filter may comprise a pore that retains or rejects a particle with a size between about 0.5 micron and about 100 microns. In some embodiments, the filter may comprise a pore that retains or rejects a particle with a size between about 0.5 micron and about 95 microns. In some embodiments, the filter may comprise a pore that retains or rejects a particle with a size between about 0.5 micron and about 90 microns. In some embodiments, the filter may comprise a pore that retains or rejects a particle with a size between about 0.5 micron and about 85 microns. In some embodiments, the filter may comprise a pore that retains or rejects a particle with a size between about 0.5 micron and about 80 microns. In some embodiments, the filter may comprise a pore that retains or rejects a particle with a size between about 0.5 micron and about 75 microns. In some embodiments, the filter may comprise a pore that retains or rejects a particle with a size between about 0.5 micron and about 70 microns. In some embodiments, the filter may comprise a pore that retains or rejects a particle with a size between about 0.5 micron and about 65 microns. In some embodiments, the filter may comprise a pore that retains or rejects a particle with a size between about 0.5 micron and about 60 microns. In some embodiments, the filter may comprise a pore that retains or rejects a particle with a size between about 0.5 micron and about 55 microns. In some embodiments, the filter may comprise a pore that retains or rejects a particle with a size between about 0.5 micron and about 50 microns. In some embodiments, the filter may comprise a pore that retains or rejects a particle with a size between about 0.5 micron and about 45 microns. In some embodiments, the filter may comprise a pore that retains or rejects a particle with a size between about 0.5 micron and about 40 microns. In some embodiments, the filter may comprise a pore that retains or rejects a particle with a size between about 0.5 micron and about 35 microns. In some embodiments, the filter may comprise a pore that retains or rejects a particle with a size between about 0.5 micron and about 30 microns. In some embodiments, the filter may comprise a pore that retains or rejects a particle with a size between about 0.5 micron and about 25 microns. In some embodiments, the filter may comprise a pore that retains or rejects a particle with a size between about 0.5 micron and about 20 microns. In some embodiments, the filter may comprise a pore that retains or rejects a particle with a size between about 0.5 micron and about 15 microns. In some embodiments, the filter may comprise a pore that retains or rejects a particle with a size between about 0.5 micron and about 10 microns. In some embodiments, the filter may comprise a pore that retains or rejects a particle with a size between about 0.5 micron and about 5 microns.

In some embodiments, the filter may comprise a pore that retains or rejects a particle with a size between about 0.6 micron and about 100 microns. In some embodiments, the filter may comprise a pore that retains or rejects a particle with a size between about 0.6 micron and about 95 microns. In some embodiments, the filter may comprise a pore that retains or rejects a particle with a size between about 0.6 micron and about 90 microns. In some embodiments, the filter may comprise a pore that retains or rejects a particle with a size between about 0.6 micron and about 85 microns. In some embodiments, the filter may comprise a pore that retains or rejects a particle with a size between about 0.6 micron and about 80 microns. In some embodiments, the filter may comprise a pore that retains or rejects a particle with a size between about 0.6 micron and about 75 microns. In some embodiments, the filter may comprise a pore that retains or rejects a particle with a size between about 0.6 micron and about 70 microns. In some embodiments, the filter may comprise a pore that retains or rejects a particle with a size between about 0.6 micron and about 65 microns. In some embodiments, the filter may comprise a pore that retains or rejects a particle with a size between about 0.6 micron and about 60 microns. In some embodiments, the filter may comprise a pore that retains or rejects a particle with a size between about 0.6 micron and about 55 microns. In some embodiments, the filter may comprise a pore that retains or rejects a particle with a size between about 0.6 micron and about 50 microns. In some embodiments, the filter may comprise a pore that retains or rejects a particle with a size between about 0.6 micron and about 45 microns. In some embodiments, the filter may comprise a pore that retains or rejects a particle with a size between about 0.6 micron and about 40 microns. In some embodiments, the filter may comprise a pore that retains or rejects a particle with a size between about 0.6 micron and about 35 microns. In some embodiments, the filter may comprise a pore that retains or rejects a particle with a size between about 0.16 micron and about 30 microns. In some embodiments, the filter may comprise a pore that retains or rejects a particle with a size between about 0.6 micron and about 25 microns. In some embodiments, the filter may comprise a pore that retains or rejects a particle with a size between about 0.6 micron and about 20 microns. In some embodiments, the filter may comprise a pore that retains or rejects a particle with a size between about 0.6 micron and about 15 microns. In some embodiments, the filter may comprise a pore that retains or rejects a particle with a size between about 0.6 micron and about 10 microns. In some embodiments, the filter may comprise a pore that retains or rejects a particle with a size between about 0.6 micron and about 5 microns.

In some embodiments, the filter may comprise a pore that retains or rejects a particle with a size between about 0.7 micron and about 100 microns. In some embodiments, the filter may comprise a pore that retains or rejects a particle with a size between about 0.7 micron and about 95 microns. In some embodiments, the filter may comprise a pore that retains or rejects a particle with a size between about 0.7 micron and about 90 microns. In some embodiments, the filter may comprise a pore that retains or rejects a particle with a size between about 0.7 micron and about 85 microns. In some embodiments, the filter may comprise a pore that retains or rejects a particle with a size between about 0.7 micron and about 80 microns. In some embodiments, the filter may comprise a pore that retains or rejects a particle with a size between about 0.7 micron and about 75 microns. In some embodiments, the filter may comprise a pore that retains or rejects a particle with a size between about 0.7 micron and about 70 microns. In some embodiments, the filter may comprise a pore that retains or rejects a particle with a size between about 0.7 micron and about 65 microns. In some embodiments, the filter may comprise a pore that retains or rejects a particle with a size between about 0.7 micron and about 60 microns. In some embodiments, the filter may comprise a pore that retains or rejects a particle with a size between about 0.7 micron and about 55 microns. In some embodiments, the filter may comprise a pore that retains or rejects a particle with a size between about 0.7 micron and about 50 microns. In some embodiments, the filter may comprise a pore that retains or rejects a particle with a size between about 0.7 micron and about 45 microns. In some embodiments, the filter may comprise a pore that retains or rejects a particle with a size between about 0.7 micron and about 40 microns. In some embodiments, the filter may comprise a pore that retains or rejects a particle with a size between about 0.7 micron and about 35 microns. In some embodiments, the filter may comprise a pore that retains or rejects a particle with a size between about 0.7 micron and about 30 microns. In some embodiments, the filter may comprise a pore that retains or rejects a particle with a size between about 0.7 micron and about 25 microns. In some embodiments, the filter may comprise a pore that retains or rejects a particle with a size between about 0.7 micron and about 20 microns. In some embodiments, the filter may comprise a pore that retains or rejects a particle with a size between about 0.7 micron and about 15 microns. In some embodiments, the filter may comprise a pore that retains or rejects a particle with a size between about 0.7 micron and about 10 microns. In some embodiments, the filter may comprise a pore that retains or rejects a particle with a size between about 0.7 micron and about 5 microns.

In some embodiments, the filter may comprise a pore that retains or rejects a particle with a size between about 0.8 micron and about 100 microns. In some embodiments, the filter may comprise a pore that retains or rejects a particle with a size between about 0.8 micron and about 95 microns. In some embodiments, the filter may comprise a pore that retains or rejects a particle with a size between about 0.8 micron and about 90 microns. In some embodiments, the filter may comprise a pore that retains or rejects a particle with a size between about 0.8 micron and about 85 microns. In some embodiments, the filter may comprise a pore that retains or rejects a particle with a size between about 0.8 micron and about 80 microns. In some embodiments, the filter may comprise a pore that retains or rejects a particle with a size between about 0.8 micron and about 75 microns. In some embodiments, the filter may comprise a pore that retains or rejects a particle with a size between about 0.8 micron and about 70 microns. In some embodiments, the filter may comprise a pore that retains or rejects a particle with a size between about 0.8 micron and about 65 microns. In some embodiments, the filter may comprise a pore that retains or rejects a particle with a size between about 0.8 micron and about 60 microns. In some embodiments, the filter may comprise a pore that retains or rejects a particle with a size between about 0.8 micron and about 55 microns. In some embodiments, the filter may comprise a pore that retains or rejects a particle with a size between about 0.8 micron and about 50 microns. In some embodiments, the filter may comprise a pore that retains or rejects a particle with a size between about 0.8 micron and about 45 microns. In some embodiments, the filter may comprise a pore that retains or rejects a particle with a size between about 0.8 micron and about 40 microns. In some embodiments, the filter may comprise a pore that retains or rejects a particle with a size between about 0.8 micron and about 35 microns. In some embodiments, the filter may comprise a pore that retains or rejects a particle with a size between about 0.8 micron and about 30 microns. In some embodiments, the filter may comprise a pore that retains or rejects a particle with a size between about 0.8 micron and about 25 microns. In some embodiments, the filter may comprise a pore that retains or rejects a particle with a size between about 0.8 micron and about 20 microns. In some embodiments, the filter may comprise a pore that retains or rejects a particle with a size between about 0.8 micron and about 15 microns. In some embodiments, the filter may comprise a pore that retains or rejects a particle with a size between about 0.8 micron and about 10 microns. In some embodiments, the filter may comprise a pore that retains or rejects a particle with a size between about 0.8 micron and about 5 microns.

In some embodiments, the filter may comprise a pore that retains or rejects a particle with a size between about 0.9 micron and about 100 microns. In some embodiments, the filter may comprise a pore that retains or rejects a particle with a size between about 0.9 micron and about 95 microns. In some embodiments, the filter may comprise a pore that retains or rejects a particle with a size between about 0.9 micron and about 90 microns. In some embodiments, the filter may comprise a pore that retains or rejects a particle with a size between about 0.9 micron and about 85 microns. In some embodiments, the filter may comprise a pore that retains or rejects a particle with a size between about 0.9 micron and about 80 microns. In some embodiments, the filter may comprise a pore that retains or rejects a particle with a size between about 0.9 micron and about 75 microns. In some embodiments, the filter may comprise a pore that retains or rejects a particle with a size between about 0.9 micron and about 70 microns. In some embodiments, the filter may comprise a pore that retains or rejects a particle with a size between about 0.9 micron and about 65 microns. In some embodiments, the filter may comprise a pore that retains or rejects a particle with a size between about 0.9 micron and about 60 microns. In some embodiments, the filter may comprise a pore that retains or rejects a particle with a size between about 0.9 micron and about 55 microns. In some embodiments, the filter may comprise a pore that retains or rejects a particle with a size between about 0.9 micron and about 50 microns. In some embodiments, the filter may comprise a pore that retains or rejects a particle with a size between about 0.9 micron and about 45 microns. In some embodiments, the filter may comprise a pore that retains or rejects a particle with a size between about 0.9 micron and about 40 microns. In some embodiments, the filter may comprise a pore that retains or rejects a particle with a size between about 0.9 micron and about 35 microns. In some embodiments, the filter may comprise a pore that retains or rejects a particle with a size between about 0.9 micron and about 30 microns. In some embodiments, the filter may comprise a pore that retains or rejects a particle with a size between about 0.9 micron and about 25 microns. In some embodiments, the filter may comprise a pore that retains or rejects a particle with a size between about 0.9 micron and about 20 microns. In some embodiments, the filter may comprise a pore that retains or rejects a particle with a size between about 0.9 micron and about 15 microns. In some embodiments, the filter may comprise a pore that retains or rejects a particle with a size between about 0.9 micron and about 10 microns. In some embodiments, the filter may comprise a pore that retains or rejects a particle with a size between about 0.9 micron and about 5 microns.

In some embodiments, the filter may comprise a surface filter. In some embodiments, the filter may comprise a depth filter. In some embodiments, the filter may comprise a filter comprising a sterilization-grade membrane. In some embodiments, the filter comprising a sterilization-grade membrane may retain a particle with a size greater than about 0.1 micron. In some embodiments, the filter comprising a sterilization-grade membrane may retain a particle with a size greater than about 0.2 micron. In some embodiments, the filter comprising a sterilization-grade membrane may retain a particle with a size greater than about 0.4 micron. In some embodiments, the step of passing of the CFPS method disclosed herein may be carried out using one or more selected from the group comprising the following: cross-flow filtration, belt filtration, microfiltration, ultrafiltration, microporous filtration, vacuum-drum filtration, sieving, membrane filtration, sand filtration, and screen filtration. In some embodiments, the depth filter may comprise a medium. In some embodiments, the medium of the depth filter may form a flow channel. In some embodiments, the medium of the depth filter may comprise a complex, tortuous flow channel In some embodiments, the medium of the depth filter may comprise two fibers. In some embodiments, the medium of the depth filter may be positively charged. In some embodiments, the medium of the depth filter may be neutrally charged. In some embodiments, the medium of the depth filter may be negatively charged. In some embodiments, the two fibers may be bonded or fixed to provide a matrix. In some embodiments, the two fibers may be bonded or fixed to provide a random matrix. In some embodiments, the two fibers may be bonded by a binder. In some embodiments, the binder may comprise a charged resin, or an uncharged resin. In some embodiments, the two fibers may absorb or entrap a particle in the cellular lysate. In some embodiments, one of the two fibers may comprise a cellulose fiber. In some embodiments, a particle in the cellular lysate may be separated through entrapment by the medium. In some embodiments, a particle in the cellular lysate may be separated through adsorption to the medium. In some embodiments, a particle in the cellular lysate may be retained in the filter through size exclusion. In some embodiments, a particle in the cellular lysate may be retained in the filter by adsorption to the medium of the filter through a hydrophobic interaction, an ionic interaction, or other types of interactions. In some embodiments, the depth filter may comprise a filter aid. In some embodiments, the filter aid may comprise diatomaceous earth. In some embodiments, the depth filter may comprise a pore that retains a particle with a size that is larger than the size of the pore. In some embodiments, the depth filter may comprise a pore that retains a particle with a size that is smaller than the size of the pore. In some embodiments, the depth filter may comprise a single-use filter. In some embodiments, the depth filter may comprise one or more selected from the group comprising a lenticular filter, a deep bed filter, a pad filter, a panel filter, and a cartridge-type depth filter.

In some embodiments, the filter may comprise three filters, a first filter, a second filter, and a third filter. In some embodiments, the first filter may comprise a filter disclosed herein. In some embodiments, the first filter may comprise a filter comprising a pore that that retains or rejects a particle with a size between about 0.5 micron and about 15 microns. In some embodiments, the second filter may comprise a filter disclosed herein. In some embodiments, the second filter may comprise a filter comprising a pore that retains or rejects a particle with a size between about 0.2 micron and about 0.5 micron. In some embodiments, the third filter may comprise a filter disclosed herein. In some embodiments, the third filter may comprise a filter comprising a pore that retains or rejects a particle with a size of about 0.2 micron. In some embodiments, at least two of the first filter, second filter and third filter may be arranged in series. In some embodiments, all three of the first filter, second filter and third filter may be arranged in series. In some embodiments, at least two of the first filter, second filter and third filter may be arranged in parallel. In some embodiments, all three of the first filter, second filter and third filter may be arranged in parallel.

In some embodiments, the filter may comprise three filters, a first depth filter, a second depth filter, and a third filter comprising a sterilization-grade membrane. In some embodiments, the first depth filter may comprise a pore that retains or rejects a particle with a size between about 0.5 micron and about 15 microns. In some embodiments, the second depth filter may comprise a pore that retains or rejects a particle with a size between about 0.2 micron and about 0.5 micron. In some embodiments, the third filter comprising the sterilization-grade membrane may comprise a pore that retains or reject a particle with a size of about 0.2 micron. In some embodiments, at least two of the first depth filter, second depth filter, and third filter comprising the sterilization-grade membrane, may be arranged in series. In some embodiments, all three of the first depth filter, second depth filter, and third filter comprising the sterilization-grade membrane, may be arranged in series. In some embodiments, at least two of the first depth filter, second depth filter, and third filter comprising the sterilization-grade membrane, may be arranged in parallel. In some embodiments, all three of the first depth filter, second depth filter and third filter comprising the sterilization-grade membrane may be arranged in parallel. When all three of the first depth filter, second depth filter, and third filter comprising the sterilization-grade membrane, are arranged in series, the step of the passing of the method disclosed herein may comprise passing the cellular lysate, first, through the first depth filter to produce a first cellular extract, then, through the second depth filter to produce a second cellular extract, and, last, through the third filter comprising the sterilization-grade membrane to produce a third cellular extract.

In some embodiments, based on the solid content in the cellular lysate, the step of the passing may be configured accordingly to optimize the extraction. In some embodiments, the step of passing may involve step-wise filtration. In some embodiments, the cellular lysate may be filtered through at least two filters arranged in series and with decreasing pore sizes. In some embodiments, the composition of a filter media may be configured for better flow, particle capture, and cellular extract quality. For example, the filter may comprise two filters, a first filter and a second filter. In some embodiments, the first filter may be used to produce a first clarified cellular lysate which then passes through the second filter which produces a second clarified cellular lysate, or a clarified cellular extract. In some embodiments, the first filter may comprise a pore that rejects or retains a particle that is larger than about 100 microns, about 50 microns, about 30 microns, or about 15 microns. In some embodiments, the second filter may comprise a pore that rejects or retains a particle that is larger than about 5 microns, about 2 microns, about 1 micron, about 500 nm, or about 200 nm. In some embodiments, the cellular lysate may be filtered through at least two filters arranged in parallel to make the filtration process resistant to clogging and increase filter surface area. In some embodiments, each of the at least two filters arranged in parallel may comprise an identical filter. In some embodiments, the at least two filters comprising identical filters may be contacted with a cellular lysate simultaneously so that the filtration process may continue even if one of the at least two filters clogs.

In some embodiments, the method may comprise a post-filtration processing step for the cellular extract depending on a property of the cellular lysate. In some embodiments, the post-filtration processing step may comprise desalting the cellular extract. In some embodiments, the post-filtration processing step may comprise subjecting the cellular extract to buffer-exchange. In some embodiments, the post-filtration processing step may comprise dialyzing the cellular extract. In some embodiments, the post-filtration processing step may comprise subjecting the cellular extract to diafiltration. In some embodiments, the post-filtration processing step may comprise freezing the cellular extract. In some embodiments, the post-filtration processing step may comprise freeze-drying the cellular extract.

In some embodiments, the method may comprise a step of contacting the cellular extract with a nucleic acid encoding a polypeptide to produce a reaction mixture. In some embodiments, the cellular extract is not subject to clarification through centrifugation prior to the step of the contacting. In some embodiments, the reaction mixture is not subject to clarification through centrifugation. In some embodiments, the nucleic acid may comprise a RNA translation template. In some embodiments, the nucleic acid may comprise an exogenous RNA translation template. In some embodiments, the reaction mixture may comprise a DNA expression template. In some embodiments, the DNA expression template may encode an open reading frame of the polypeptide. In some embodiments, the open reading frame may be operatively linked to a promoter for a DNA-dependent RNA polymerase. In some embodiments, the DNA-dependent RNA polymerase may direct transcription of the DNA expression template.

In some embodiments, the contacting may comprise adding to the cellular extract the nucleic acid encoding the polypeptide. In some embodiments, the reaction mixture may comprise an amino acid, a nucleotide, a salt, a cofactor, an energy source, a translation template, or a combination thereof. In some embodiments, the reaction mixture may comprise a divalent cation. In some embodiments, the reaction mixture may comprise any component disclosed herein. In some embodiments, the contacting may comprise adding to the cellular extract one or more selected from the group comprising an amino acid, a nucleotide, a salt, a cofactor, an energy source, and a translation template. In some embodiments, the energy source may comprise a phosphate group. In some embodiments, the energy source may comprise a non-phosphorylated energy group. The energy source may comprise one or more selected from phosphoenolpyruvate, glutamate, glycerol, pyruvate, glucose, and creatine phosphate. In some embodiments, the nucleotide in the reaction mixture may comprise a nucleoside monophosphate (NMP), a nucleoside diphosphate (NDP), a nucleoside triphosphate (NTP), or a combination thereof. In some embodiments, the reaction mixture may comprise a transfer RNA (tRNA). In some embodiments, the reaction mixture may comprise an enzyme cofactor. In some embodiments, the reaction mixture may comprise a metabolite. In some embodiments, the metabolite may comprise one or more selected from the group comprising folinic acid, nicotinamide adenine dinucleotide, coenzyme-A, and sodium oxalate.

In some embodiments, the reaction mixture may comprise an organic anion. In some embodiments, the organic anion may comprise glutamate, acetate, or a combination thereof. In some embodiments, the organic anion may be present at a concentration between about 0 mM and about 300 mM, e.g., about 0.001 mM, 0.002 mM, 0.003 mM, 0.004 mM, 0.005 mM, 0.006 mM, 0.007 mM, 0.008 mM, 0.009 mM, 0.01 mM, 0.011 mM, 0.012 mM, 0.013 mM, 0.014 mM, 0.015 mM, 0.016 mM, 0.017 mM, 0.018 mM, 0.019 mM, 0.02 mM, 0.021 mM, 0.022 mM, 0.023 mM, 0.024 mM, 0.025 mM, 0.026 mM, 0.027 mM, 0.028 mM, 0.029 mM, 0.03 mM, 0.031 mM, 0.032 mM, 0.033 mM, 0.034 mM, 0.035 mM, 0.036 mM, 0.037 mM, 0.038 mM, 0.039 mM, 0.04 mM, 0.041 mM, 0.042 mM, 0.043 mM, 0.044 mM, 0.045 mM, 0.046 mM, 0.047 mM, 0.048 mM, 0.049 mM, 0.05 mM, 0.055 mM, 0.06 mM, 0.065 mM, 0.07 mM, 0.075 mM, 0.08 mM, 0.085 mM, 0.09 mM, 0.095 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, or about 300 mM, or any concentration therebetween.

In some embodiments, the reaction mixture may comprise a halide anion. In some embodiments, the halide anion may be present at a concentration between about 0 mM and about 300 mM, e.g., about 0.001 mM, 0.002 mM, 0.003 mM, 0.004 mM, 0.005 mM, 0.006 mM, 0.007 mM, 0.008 mM, 0.009 mM, 0.01 mM, 0.011 mM, 0.012 mM, 0.013 mM, 0.014 mM, 0.015 mM, 0.016 mM, 0.017 mM, 0.018 mM, 0.019 mM, 0.02 mM, 0.021 mM, 0.022 mM, 0.023 mM, 0.024 mM, 0.025 mM, 0.026 mM, 0.027 mM, 0.028 mM, 0.029 mM, 0.03 mM, 0.031 mM, 0.032 mM, 0.033 mM, 0.034 mM, 0.035 mM, 0.036 mM, 0.037 mM, 0.038 mM, 0.039 mM, 0.04 mM, 0.041 mM, 0.042 mM, 0.043 mM, 0.044 mM, 0.045 mM, 0.046 mM, 0.047 mM, 0.048 mM, 0.049 mM, 0.05 mM, 0.055 mM, 0.06 mM, 0.065 mM, 0.07 mM, 0.075 mM, 0.08 mM, 0.085 mM, 0.09 mM, 0.095 mM, 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, or about 300 mM, or any concentration therebetween.

In some embodiments, the reaction mixture may comprise an organic cation. In some embodiments, the organic cation may comprise a polyamine. In some embodiments, the polyamine may comprise spermidine, putrescine, or a combination thereof. In some embodiments, the organic cation may be present at a concentration between about 0 mM and about 3 mM, e.g., about 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 21 nM, 22 nM, 23 nM, 24 nM, 25 nM, 26 nM, 27 nM, 28 nM, 29 nM, 30 nM, 31 nM, 32 nM, 33 nM, 34 nM, 35 nM, 36 nM, 37 nM, 38 nM, 39 nM, 40 nM, 41 nM, 42 nM, 43 nM, 44 nM, 45 nM, 46 nM, 47 nM, 48 nM, 49 nM, 50 nM, 51 nM, 52 nM, 53 nM, 54 nM, 55 nM, 56 nM, 57 nM, 58 nM, 59 nM, 60 nM, 61 nM, 62 nM, 63 nM, 64 nM, 65 nM, 66 nM, 67 nM, 68 nM, 69 nM, 70 nM, 71 nM, 72 nM, 73 nM, 74 nM, 75 nM, 76 nM, 77 nM, 78 nM, 79 nM, 80 nM, 81 nM, 82 nM, 83 nM, 84 nM, 85 nM, 86 nM, 87 nM, 88 nM, 89 nM, 90 nM, 91 nM, 92 nM, 93 nM, 94 nM, 95 nM, 96 nM, 97 nM, 98 nM, 99 nM, 100 nM, 110 nM, 120 nM, 130 nM, 140 nM, 150 nM, 160 nM, 170 nM, 180 nM, 190 nM, 200 nM, 210 nM, 220 nM, 230 nM, 240 nM, 250 nM, 260 nM, 270 nM, 280 nM, 290 nM, 300 nM, 310 nM, 320 nM, 330 nM, 340 nM, 350 nM, 360 nM, 370 nM, 380 nM, 390 nM, 400 nM, 410 nM, 420 nM, 430 nM, 440 nM, 450 nM, 460 nM, 470 nM, 480 nM, 490 nM, 500 nM, 510 nM, 520 nM, 530 nM, 540 nM, 550 nM, 560 nM, 570 nM, 580 nM, 590 nM, 600 nM, 610 nM, 620 nM, 630 nM, 640 nM, 650 nM, 660 nM, 670 nM, 680 nM, 690 nM, 700 nM, 710 nM, 720 nM, 730 nM, 740 nM, 750 nM, 760 nM, 770 nM, 780 nM, 790 nM, 800 nM, 810 nM, 820 nM, 830 nM, 840 nM, 850 nM, 860 nM, 870 nM, 880 nM, 890 nM, 900 nM, 910 nM, 920 nM, 930 nM, 940 nM, 950 nM, 960 nM, 970 nM, 980 nM, 990 nM, 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1000 μM, 1.05 mM, 1.1 mM, 1.15 mM, 1.2 mM, 1.25 mM, 1.3 mM, 1.35 mM, 1.4 mM, 1.45 mM, 1.5 mM, 1.55 mM, 1.6 mM, 1.65 mM, 1.7 mM, 1.75 mM, 1.8 mM, 1.85 mM, 1.9 mM, 1.95 mM, 2 mM, 2.05 mM, 2.1 mM, 2.15 mM, 2.2 mM, 2.25 mM, 2.3 mM, 2.35 mM, 2.4 mM, 2.45 mM, 2.5 mM, 2.55 mM, 2.6 mM, 2.65 mM, 2.7 mM, 2.75 mM, 2.8 mM, 2.85 mM, 2.9 mM, 2.95 mM, or about 3 mM, or any concentration therebetween.

In some embodiments, the reaction mixture may comprise an inorganic cation. In some embodiments, the inorganic cation may comprise a monovalent cation. In some embodiments, the monovalent cation may comprise sodium, potassium, lithium, or a combination thereof. In some embodiments, the inorganic cation may comprise a divalent cation. In some embodiments, the divalent cation may comprise magnesium, calcium, manganese, or a combination thereof. In some embodiments, the divalent cation comprises magnesium. In some embodiments, the magnesium may be present at a concentration between about 1 mM and about 50 mM, e.g., about 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, 20 mM, 20.1 mM, 20.2 mM, 20.3 mM, 20.4 mM, 20.5 mM, 20.6 mM, 20.7 mM, 20.8 mM, 20.9 mM, 21 mM, 21.1 mM, 21.2 mM, 21.3 mM, 21.4 mM, 21.5 mM, 21.6 mM, 21.7 mM, 21.8 mM, 21.9 mM, 22 mM, 22.1 mM, 22.2 mM, 22.3 mM, 22.4 mM, 22.5 mM, 22.6 mM, 22.7 mM, 22.8 mM, 22.9 mM, 23 mM, 23.1 mM, 23.2 mM, 23.3 mM, 23.4 mM, 23.5 mM, 23.6 mM, 23.7 mM, 23.8 mM, 23.9 mM, 24 mM, 24.1 mM, 24.2 mM, 24.3 mM, 24.4 mM, 24.5 mM, 24.6 mM, 24.7 mM, 24.8 mM, 24.9 mM, 25 mM, 25.1 mM, 25.2 mM, 25.3 mM, 25.4 mM, 25.5 mM, 25.6 mM, 25.7 mM, 25.8 mM, 25.9 mM, 26 mM, 26.1 mM, 26.2 mM, 26.3 mM, 26.4 mM, 26.5 mM, 26.6 mM, 26.7 mM, 26.8 mM, 26.9 mM, 27 mM, 27.1 mM, 27.2 mM, 27.3 mM, 27.4 mM, 27.5 mM, 27.6 mM, 27.7 mM, 27.8 mM, 27.9 mM, 28 mM, 28.1 mM, 28.2 mM, 28.3 mM, 28.4 mM, 28.5 mM, 28.6 mM, 28.7 mM, 28.8 mM, 28.9 mM, 29 mM, 29.1 mM, 29.2 mM, 29.3 mM, 29.4 mM, 29.5 mM, 29.6 mM, 29.7 mM, 29.8 mM, 29.9 mM, 30 mM, 30.1 mM, 30.2 mM, 30.3 mM, 30.4 mM, 30.5 mM, 30.6 mM, 30.7 mM, 30.8 mM, 30.9 mM, 31 mM, 31.1 mM, 31.2 mM, 31.3 mM, 31.4 mM, 31.5 mM, 31.6 mM, 31.7 mM, 31.8 mM, 31.9 mM, 32 mM, 32.1 mM, 32.2 mM, 32.3 mM, 32.4 mM, 32.5 mM, 32.6 mM, 32.7 mM, 32.8 mM, 32.9 mM, 33 mM, 33.1 mM, 33.2 mM, 33.3 mM, 33.4 mM, 33.5 mM, 33.6 mM, 33.7 mM, 33.8 mM, 33.9 mM, 34 mM, 34.1 mM, 34.2 mM, 34.3 mM, 34.4 mM, 34.5 mM, 34.6 mM, 34.7 mM, 34.8 mM, 34.9 mM, 35 mM, 35.1 mM, 35.2 mM, 35.3 mM, 35.4 mM, 35.5 mM, 35.6 mM, 35.7 mM, 35.8 mM, 35.9 mM, 36 mM, 36.1 mM, 36.2 mM, 36.3 mM, 36.4 mM, 36.5 mM, 36.6 mM, 36.7 mM, 36.8 mM, 36.9 mM, 37 mM, 37.1 mM, 37.2 mM, 37.3 mM, 37.4 mM, 37.5 mM, 37.6 mM, 37.7 mM, 37.8 mM, 37.9 mM, 38 mM, 38.1 mM, 38.2 mM, 38.3 mM, 38.4 mM, 38.5 mM, 38.6 mM, 38.7 mM, 38.8 mM, 38.9 mM, 39 mM, 39.1 mM, 39.2 mM, 39.3 mM, 39.4 mM, 39.5 mM, 39.6 mM, 39.7 mM, 39.8 mM, 39.9 mM, 40 mM, 40.1 mM, 40.2 mM, 40.3 mM, 40.4 mM, 40.5 mM, 40.6 mM, 40.7 mM, 40.8 mM, 40.9 mM, 41 mM, 41.1 mM, 41.2 mM, 41.3 mM, 41.4 mM, 41.5 mM, 41.6 mM, 41.7 mM, 41.8 mM, 41.9 mM, 42 mM, 42.1 mM, 42.2 mM, 42.3 mM, 42.4 mM, 42.5 mM, 42.6 mM, 42.7 mM, 42.8 mM, 42.9 mM, 43 mM, 43.1 mM, 43.2 mM, 43.3 mM, 43.4 mM, 43.5 mM, 43.6 mM, 43.7 mM, 43.8 mM, 43.9 mM, 44 mM, 44.1 mM, 44.2 mM, 44.3 mM, 44.4 mM, 44.5 mM, 44.6 mM, 44.7 mM, 44.8 mM, 44.9 mM, 45 mM, 45.1 mM, 45.2 mM, 45.3 mM, 45.4 mM, 45.5 mM, 45.6 mM, 45.7 mM, 45.8 mM, 45.9 mM, 46 mM, 46.1 mM, 46.2 mM, 46.3 mM, 46.4 mM, 46.5 mM, 46.6 mM, 46.7 mM, 46.8 mM, 46.9 mM, 47 mM, 47.1 mM, 47.2 mM, 47.3 mM, 47.4 mM, 47.5 mM, 47.6 mM, 47.7 mM, 47.8 mM, 47.9 mM, 48 mM, 48.1 mM, 48.2 mM, 48.3 mM, 48.4 mM, 48.5 mM, 48.6 mM, 48.7 mM, 48.8 mM, 48.9 mM, 49 mM, 49.1 mM, 49.2 mM, 49.3 mM, 49.4 mM, 49.5 mM, 49.6 mM, 49.7 mM, 49.8 mM, 49.9 mM, or about 50 mM, or any concentration therebetween.

In some embodiments, the reaction mixture may comprise an endogenous nucleotide, i.e., a nucleotide, such as a nucleoside triphosphate (NTP), that is present in the cellular extract and derived from the cell. In some embodiments, the reaction mixture may comprise an exogenous nucleotide, i.e., a nucleotide, such as a nucleoside triphosphate (NTP), a nucleoside diphosphate (NDP), or a nucleoside monophosphate (NMP), that is added to the cellular extract. In some embodiments, the nucleotide may comprise a NTP, e.g., ATP, GTP, CTP, UTP, or a combination thereof. In some embodiments, the nucleotide may comprise a NMP, e.g., AMP, GMP, CMP, UMP, or a combination thereof. In some embodiments, the reaction mixture may comprise a combination of NTPs, NDPs and NMPs. For example, the reaction mixture may comprise a mixture of NTPs and NDPs, a mixture of NTPs and NMPs, a mixture of NDPs and NMPs, or mixture of NTPs, NDPs and NMPs.

In some embodiments, each NTP, e.g., ATP, GTP, CTP, or UTP, may be present in the reaction mixture at a concentration between about 0.1 mM and about 5 mM, e.g., about 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.01 mM, 1.02 mM, 1.03 mM, 1.04 mM, 1.05 mM, 1.06 mM, 1.07 mM, 1.08 mM, 1.09 mM, 1.1 mM, 1.11 mM, 1.12 mM, 1.13 mM, 1.14 mM, 1.15 mM, 1.16 mM, 1.17 mM, 1.18 mM, 1.19 mM, 1.2 mM, 1.21 mM, 1.22 mM, 1.23 mM, 1.24 mM, 1.25 mM, 1.26 mM, 1.27 mM, 1.28 mM, 1.29 mM, 1.3 mM, 1.31 mM, 1.32 mM, 1.33 mM, 1.34 mM, 1.35 mM, 1.36 mM, 1.37 mM, 1.38 mM, 1.39 mM, 1.4 mM, 1.41 mM, 1.42 mM, 1.43 mM, 1.44 mM, 1.45 mM, 1.46 mM, 1.47 mM, 1.48 mM, 1.49 mM, 1.5 mM, 1.51 mM, 1.52 mM, 1.53 mM, 1.54 mM, 1.55 mM, 1.56 mM, 1.57 mM, 1.58 mM, 1.59 mM, 1.6 mM, 1.61 mM, 1.62 mM, 1.63 mM, 1.64 mM, 1.65 mM, 1.66 mM, 1.67 mM, 1.68 mM, 1.69 mM, 1.7 mM, 1.71 mM, 1.72 mM, 1.73 mM, 1.74 mM, 1.75 mM, 1.76 mM, 1.77 mM, 1.78 mM, 1.79 mM, 1.8 mM, 1.81 mM, 1.82 mM, 1.83 mM, 1.84 mM, 1.85 mM, 1.86 mM, 1.87 mM, 1.88 mM, 1.89 mM, 1.9 mM, 1.91 mM, 1.92 mM, 1.93 mM, 1.94 mM, 1.95 mM, 1.96 mM, 1.97 mM, 1.98 mM, 1.99 mM, 2 mM, 2.01 mM, 2.02 mM, 2.03 mM, 2.04 mM, 2.05 mM, 2.06 mM, 2.07 mM, 2.08 mM, 2.09 mM, 2.1 mM, 2.11 mM, 2.12 mM, 2.13 mM, 2.14 mM, 2.15 mM, 2.16 mM, 2.17 mM, 2.18 mM, 2.19 mM, 2.2 mM, 2.21 mM, 2.22 mM, 2.23 mM, 2.24 mM, 2.25 mM, 2.26 mM, 2.27 mM, 2.28 mM, 2.29 mM, 2.3 mM, 2.31 mM, 2.32 mM, 2.33 mM, 2.34 mM, 2.35 mM, 2.36 mM, 2.37 mM, 2.38 mM, 2.39 mM, 2.4 mM, 2.41 mM, 2.42 mM, 2.43 mM, 2.44 mM, 2.45 mM, 2.46 mM, 2.47 mM, 2.48 mM, 2.49 mM, 2.5 mM, 2.51 mM, 2.52 mM, 2.53 mM, 2.54 mM, 2.55 mM, 2.56 mM, 2.57 mM, 2.58 mM, 2.59 mM, 2.6 mM, 2.61 mM, 2.62 mM, 2.63 mM, 2.64 mM, 2.65 mM, 2.66 mM, 2.67 mM, 2.68 mM, 2.69 mM, 2.7 mM, 2.71 mM, 2.72 mM, 2.73 mM, 2.74 mM, 2.75 mM, 2.76 mM, 2.77 mM, 2.78 mM, 2.79 mM, 2.8 mM, 2.81 mM, 2.82 mM, 2.83 mM, 2.84 mM, 2.85 mM, 2.86 mM, 2.87 mM, 2.88 mM, 2.89 mM, 2.9 mM, 2.91 mM, 2.92 mM, 2.93 mM, 2.94 mM, 2.95 mM, 2.96 mM, 2.97 mM, 2.98 mM, 2.99 mM, 3 mM, 3.01 mM, 3.02 mM, 3.03 mM, 3.04 mM, 3.05 mM, 3.06 mM, 3.07 mM, 3.08 mM, 3.09 mM, 3.1 mM, 3.11 mM, 3.12 mM, 3.13 mM, 3.14 mM, 3.15 mM, 3.16 mM, 3.17 mM, 3.18 mM, 3.19 mM, 3.2 mM, 3.21 mM, 3.22 mM, 3.23 mM, 3.24 mM, 3.25 mM, 3.26 mM, 3.27 mM, 3.28 mM, 3.29 mM, 3.3 mM, 3.31 mM, 3.32 mM, 3.33 mM, 3.34 mM, 3.35 mM, 3.36 mM, 3.37 mM, 3.38 mM, 3.39 mM, 3.4 mM, 3.41 mM, 3.42 mM, 3.43 mM, 3.44 mM, 3.45 mM, 3.46 mM, 3.47 mM, 3.48 mM, 3.49 mM, 3.5 mM, 3.51 mM, 3.52 mM, 3.53 mM, 3.54 mM, 3.55 mM, 3.56 mM, 3.57 mM, 3.58 mM, 3.59 mM, 3.6 mM, 3.61 mM, 3.62 mM, 3.63 mM, 3.64 mM, 3.65 mM, 3.66 mM, 3.67 mM, 3.68 mM, 3.69 mM, 3.7 mM, 3.71 mM, 3.72 mM, 3.73 mM, 3.74 mM, 3.75 mM, 3.76 mM, 3.77 mM, 3.78 mM, 3.79 mM, 3.8 mM, 3.81 mM, 3.82 mM, 3.83 mM, 3.84 mM, 3.85 mM, 3.86 mM, 3.87 mM, 3.88 mM, 3.89 mM, 3.9 mM, 3.91 mM, 3.92 mM, 3.93 mM, 3.94 mM, 3.95 mM, 3.96 mM, 3.97 mM, 3.98 mM, 3.99 mM, 4 mM, 4.01 mM, 4.02 mM, 4.03 mM, 4.04 mM, 4.05 mM, 4.06 mM, 4.07 mM, 4.08 mM, 4.09 mM, 4.1 mM, 4.11 mM, 4.12 mM, 4.13 mM, 4.14 mM, 4.15 mM, 4.16 mM, 4.17 mM, 4.18 mM, 4.19 mM, 4.2 mM, 4.21 mM, 4.22 mM, 4.23 mM, 4.24 mM, 4.25 mM, 4.26 mM, 4.27 mM, 4.28 mM, 4.29 mM, 4.3 mM, 4.31 mM, 4.32 mM, 4.33 mM, 4.34 mM, 4.35 mM, 4.36 mM, 4.37 mM, 4.38 mM, 4.39 mM, 4.4 mM, 4.41 mM, 4.42 mM, 4.43 mM, 4.44 mM, 4.45 mM, 4.46 mM, 4.47 mM, 4.48 mM, 4.49 mM, 4.5 mM, 4.51 mM, 4.52 mM, 4.53 mM, 4.54 mM, 4.55 mM, 4.56 mM, 4.57 mM, 4.58 mM, 4.59 mM, 4.6 mM, 4.61 mM, 4.62 mM, 4.63 mM, 4.64 mM, 4.65 mM, 4.66 mM, 4.67 mM, 4.68 mM, 4.69 mM, 4.7 mM, 4.71 mM, 4.72 mM, 4.73 mM, 4.74 mM, 4.75 mM, 4.76 mM, 4.77 mM, 4.78 mM, 4.79 mM, 4.8 mM, 4.81 mM, 4.82 mM, 4.83 mM, 4.84 mM, 4.85 mM, 4.86 mM, 4.87 mM, 4.88 mM, 4.89 mM, 4.9 mM, 4.91 mM, 4.92 mM, 4.93 mM, 4.94 mM, 4.95 mM, 4.96 mM, 4.97 mM, 4.98 mM, 4.99 mM, or about 5 mM, or any concentration therebetween.

In some embodiments, each NMP, e.g., AMP, GMP, CMP, UMP, may be present in the reaction mixture at a concentration between about 0.1 mM and about 5 mM, e.g., about 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.01 mM, 1.02 mM, 1.03 mM, 1.04 mM, 1.05 mM, 1.06 mM, 1.07 mM, 1.08 mM, 1.09 mM, 1.1 mM, 1.11 mM, 1.12 mM, 1.13 mM, 1.14 mM, 1.15 mM, 1.16 mM, 1.17 mM, 1.18 mM, 1.19 mM, 1.2 mM, 1.21 mM, 1.22 mM, 1.23 mM, 1.24 mM, 1.25 mM, 1.26 mM, 1.27 mM, 1.28 mM, 1.29 mM, 1.3 mM, 1.31 mM, 1.32 mM, 1.33 mM, 1.34 mM, 1.35 mM, 1.36 mM, 1.37 mM, 1.38 mM, 1.39 mM, 1.4 mM, 1.41 mM, 1.42 mM, 1.43 mM, 1.44 mM, 1.45 mM, 1.46 mM, 1.47 mM, 1.48 mM, 1.49 mM, 1.5 mM, 1.51 mM, 1.52 mM, 1.53 mM, 1.54 mM, 1.55 mM, 1.56 mM, 1.57 mM, 1.58 mM, 1.59 mM, 1.6 mM, 1.61 mM, 1.62 mM, 1.63 mM, 1.64 mM, 1.65 mM, 1.66 mM, 1.67 mM, 1.68 mM, 1.69 mM, 1.7 mM, 1.71 mM, 1.72 mM, 1.73 mM, 1.74 mM, 1.75 mM, 1.76 mM, 1.77 mM, 1.78 mM, 1.79 mM, 1.8 mM, 1.81 mM, 1.82 mM, 1.83 mM, 1.84 mM, 1.85 mM, 1.86 mM, 1.87 mM, 1.88 mM, 1.89 mM, 1.9 mM, 1.91 mM, 1.92 mM, 1.93 mM, 1.94 mM, 1.95 mM, 1.96 mM, 1.97 mM, 1.98 mM, 1.99 mM, 2 mM, 2.01 mM, 2.02 mM, 2.03 mM, 2.04 mM, 2.05 mM, 2.06 mM, 2.07 mM, 2.08 mM, 2.09 mM, 2.1 mM, 2.11 mM, 2.12 mM, 2.13 mM, 2.14 mM, 2.15 mM, 2.16 mM, 2.17 mM, 2.18 mM, 2.19 mM, 2.2 mM, 2.21 mM, 2.22 mM, 2.23 mM, 2.24 mM, 2.25 mM, 2.26 mM, 2.27 mM, 2.28 mM, 2.29 mM, 2.3 mM, 2.31 mM, 2.32 mM, 2.33 mM, 2.34 mM, 2.35 mM, 2.36 mM, 2.37 mM, 2.38 mM, 2.39 mM, 2.4 mM, 2.41 mM, 2.42 mM, 2.43 mM, 2.44 mM, 2.45 mM, 2.46 mM, 2.47 mM, 2.48 mM, 2.49 mM, 2.5 mM, 2.51 mM, 2.52 mM, 2.53 mM, 2.54 mM, 2.55 mM, 2.56 mM, 2.57 mM, 2.58 mM, 2.59 mM, 2.6 mM, 2.61 mM, 2.62 mM, 2.63 mM, 2.64 mM, 2.65 mM, 2.66 mM, 2.67 mM, 2.68 mM, 2.69 mM, 2.7 mM, 2.71 mM, 2.72 mM, 2.73 mM, 2.74 mM, 2.75 mM, 2.76 mM, 2.77 mM, 2.78 mM, 2.79 mM, 2.8 mM, 2.81 mM, 2.82 mM, 2.83 mM, 2.84 mM, 2.85 mM, 2.86 mM, 2.87 mM, 2.88 mM, 2.89 mM, 2.9 mM, 2.91 mM, 2.92 mM, 2.93 mM, 2.94 mM, 2.95 mM, 2.96 mM, 2.97 mM, 2.98 mM, 2.99 mM, 3 mM, 3.01 mM, 3.02 mM, 3.03 mM, 3.04 mM, 3.05 mM, 3.06 mM, 3.07 mM, 3.08 mM, 3.09 mM, 3.1 mM, 3.11 mM, 3.12 mM, 3.13 mM, 3.14 mM, 3.15 mM, 3.16 mM, 3.17 mM, 3.18 mM, 3.19 mM, 3.2 mM, 3.21 mM, 3.22 mM, 3.23 mM, 3.24 mM, 3.25 mM, 3.26 mM, 3.27 mM, 3.28 mM, 3.29 mM, 3.3 mM, 3.31 mM, 3.32 mM, 3.33 mM, 3.34 mM, 3.35 mM, 3.36 mM, 3.37 mM, 3.38 mM, 3.39 mM, 3.4 mM, 3.41 mM, 3.42 mM, 3.43 mM, 3.44 mM, 3.45 mM, 3.46 mM, 3.47 mM, 3.48 mM, 3.49 mM, 3.5 mM, 3.51 mM, 3.52 mM, 3.53 mM, 3.54 mM, 3.55 mM, 3.56 mM, 3.57 mM, 3.58 mM, 3.59 mM, 3.6 mM, 3.61 mM, 3.62 mM, 3.63 mM, 3.64 mM, 3.65 mM, 3.66 mM, 3.67 mM, 3.68 mM, 3.69 mM, 3.7 mM, 3.71 mM, 3.72 mM, 3.73 mM, 3.74 mM, 3.75 mM, 3.76 mM, 3.77 mM, 3.78 mM, 3.79 mM, 3.8 mM, 3.81 mM, 3.82 mM, 3.83 mM, 3.84 mM, 3.85 mM, 3.86 mM, 3.87 mM, 3.88 mM, 3.89 mM, 3.9 mM, 3.91 mM, 3.92 mM, 3.93 mM, 3.94 mM, 3.95 mM, 3.96 mM, 3.97 mM, 3.98 mM, 3.99 mM, 4 mM, 4.01 mM, 4.02 mM, 4.03 mM, 4.04 mM, 4.05 mM, 4.06 mM, 4.07 mM, 4.08 mM, 4.09 mM, 4.1 mM, 4.11 mM, 4.12 mM, 4.13 mM, 4.14 mM, 4.15 mM, 4.16 mM, 4.17 mM, 4.18 mM, 4.19 mM, 4.2 mM, 4.21 mM, 4.22 mM, 4.23 mM, 4.24 mM, 4.25 mM, 4.26 mM, 4.27 mM, 4.28 mM, 4.29 mM, 4.3 mM, 4.31 mM, 4.32 mM, 4.33 mM, 4.34 mM, 4.35 mM, 4.36 mM, 4.37 mM, 4.38 mM, 4.39 mM, 4.4 mM, 4.41 mM, 4.42 mM, 4.43 mM, 4.44 mM, 4.45 mM, 4.46 mM, 4.47 mM, 4.48 mM, 4.49 mM, 4.5 mM, 4.51 mM, 4.52 mM, 4.53 mM, 4.54 mM, 4.55 mM, 4.56 mM, 4.57 mM, 4.58 mM, 4.59 mM, 4.6 mM, 4.61 mM, 4.62 mM, 4.63 mM, 4.64 mM, 4.65 mM, 4.66 mM, 4.67 mM, 4.68 mM, 4.69 mM, 4.7 mM, 4.71 mM, 4.72 mM, 4.73 mM, 4.74 mM, 4.75 mM, 4.76 mM, 4.77 mM, 4.78 mM, 4.79 mM, 4.8 mM, 4.81 mM, 4.82 mM, 4.83 mM, 4.84 mM, 4.85 mM, 4.86 mM, 4.87 mM, 4.88 mM, 4.89 mM, 4.9 mM, 4.91 mM, 4.92 mM, 4.93 mM, 4.94 mM, 4.95 mM, 4.96 mM, 4.97 mM, 4.98 mM, 4.99 mM, or about 5 mM, or any concentration therebetween.

In some embodiments, the reaction mixture may comprise an amino acid. In some embodiments, the amino acid may comprise an endogenous amino acid, i.e., an amino acid that is present in the cellular extract and derived from the cell. In some embodiments, the amino acid may comprise an exogenous amino acid, i.e., an amino acid that is added to the cellular extract. In some embodiments, the reaction mixture may comprise one or more canonical amino acids selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, each individual amino acid may be present in the reaction mixture at a concentration between about 0.1 mM and about 5 mM, e.g., about 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.01 mM, 1.02 mM, 1.03 mM, 1.04 mM, 1.05 mM, 1.06 mM, 1.07 mM, 1.08 mM, 1.09 mM, 1.1 mM, 1.11 mM, 1.12 mM, 1.13 mM, 1.14 mM, 1.15 mM, 1.16 mM, 1.17 mM, 1.18 mM, 1.19 mM, 1.2 mM, 1.21 mM, 1.22 mM, 1.23 mM, 1.24 mM, 1.25 mM, 1.26 mM, 1.27 mM, 1.28 mM, 1.29 mM, 1.3 mM, 1.31 mM, 1.32 mM, 1.33 mM, 1.34 mM, 1.35 mM, 1.36 mM, 1.37 mM, 1.38 mM, 1.39 mM, 1.4 mM, 1.41 mM, 1.42 mM, 1.43 mM, 1.44 mM, 1.45 mM, 1.46 mM, 1.47 mM, 1.48 mM, 1.49 mM, 1.5 mM, 1.51 mM, 1.52 mM, 1.53 mM, 1.54 mM, 1.55 mM, 1.56 mM, 1.57 mM, 1.58 mM, 1.59 mM, 1.6 mM, 1.61 mM, 1.62 mM, 1.63 mM, 1.64 mM, 1.65 mM, 1.66 mM, 1.67 mM, 1.68 mM, 1.69 mM, 1.7 mM, 1.71 mM, 1.72 mM, 1.73 mM, 1.74 mM, 1.75 mM, 1.76 mM, 1.77 mM, 1.78 mM, 1.79 mM, 1.8 mM, 1.81 mM, 1.82 mM, 1.83 mM, 1.84 mM, 1.85 mM, 1.86 mM, 1.87 mM, 1.88 mM, 1.89 mM, 1.9 mM, 1.91 mM, 1.92 mM, 1.93 mM, 1.94 mM, 1.95 mM, 1.96 mM, 1.97 mM, 1.98 mM, 1.99 mM, 2 mM, 2.01 mM, 2.02 mM, 2.03 mM, 2.04 mM, 2.05 mM, 2.06 mM, 2.07 mM, 2.08 mM, 2.09 mM, 2.1 mM, 2.11 mM, 2.12 mM, 2.13 mM, 2.14 mM, 2.15 mM, 2.16 mM, 2.17 mM, 2.18 mM, 2.19 mM, 2.2 mM, 2.21 mM, 2.22 mM, 2.23 mM, 2.24 mM, 2.25 mM, 2.26 mM, 2.27 mM, 2.28 mM, 2.29 mM, 2.3 mM, 2.31 mM, 2.32 mM, 2.33 mM, 2.34 mM, 2.35 mM, 2.36 mM, 2.37 mM, 2.38 mM, 2.39 mM, 2.4 mM, 2.41 mM, 2.42 mM, 2.43 mM, 2.44 mM, 2.45 mM, 2.46 mM, 2.47 mM, 2.48 mM, 2.49 mM, 2.5 mM, 2.51 mM, 2.52 mM, 2.53 mM, 2.54 mM, 2.55 mM, 2.56 mM, 2.57 mM, 2.58 mM, 2.59 mM, 2.6 mM, 2.61 mM, 2.62 mM, 2.63 mM, 2.64 mM, 2.65 mM, 2.66 mM, 2.67 mM, 2.68 mM, 2.69 mM, 2.7 mM, 2.71 mM, 2.72 mM, 2.73 mM, 2.74 mM, 2.75 mM, 2.76 mM, 2.77 mM, 2.78 mM, 2.79 mM, 2.8 mM, 2.81 mM, 2.82 mM, 2.83 mM, 2.84 mM, 2.85 mM, 2.86 mM, 2.87 mM, 2.88 mM, 2.89 mM, 2.9 mM, 2.91 mM, 2.92 mM, 2.93 mM, 2.94 mM, 2.95 mM, 2.96 mM, 2.97 mM, 2.98 mM, 2.99 mM, 3 mM, 3.01 mM, 3.02 mM, 3.03 mM, 3.04 mM, 3.05 mM, 3.06 mM, 3.07 mM, 3.08 mM, 3.09 mM, 3.1 mM, 3.11 mM, 3.12 mM, 3.13 mM, 3.14 mM, 3.15 mM, 3.16 mM, 3.17 mM, 3.18 mM, 3.19 mM, 3.2 mM, 3.21 mM, 3.22 mM, 3.23 mM, 3.24 mM, 3.25 mM, 3.26 mM, 3.27 mM, 3.28 mM, 3.29 mM, 3.3 mM, 3.31 mM, 3.32 mM, 3.33 mM, 3.34 mM, 3.35 mM, 3.36 mM, 3.37 mM, 3.38 mM, 3.39 mM, 3.4 mM, 3.41 mM, 3.42 mM, 3.43 mM, 3.44 mM, 3.45 mM, 3.46 mM, 3.47 mM, 3.48 mM, 3.49 mM, 3.5 mM, 3.51 mM, 3.52 mM, 3.53 mM, 3.54 mM, 3.55 mM, 3.56 mM, 3.57 mM, 3.58 mM, 3.59 mM, 3.6 mM, 3.61 mM, 3.62 mM, 3.63 mM, 3.64 mM, 3.65 mM, 3.66 mM, 3.67 mM, 3.68 mM, 3.69 mM, 3.7 mM, 3.71 mM, 3.72 mM, 3.73 mM, 3.74 mM, 3.75 mM, 3.76 mM, 3.77 mM, 3.78 mM, 3.79 mM, 3.8 mM, 3.81 mM, 3.82 mM, 3.83 mM, 3.84 mM, 3.85 mM, 3.86 mM, 3.87 mM, 3.88 mM, 3.89 mM, 3.9 mM, 3.91 mM, 3.92 mM, 3.93 mM, 3.94 mM, 3.95 mM, 3.96 mM, 3.97 mM, 3.98 mM, 3.99 mM, 4 mM, 4.01 mM, 4.02 mM, 4.03 mM, 4.04 mM, 4.05 mM, 4.06 mM, 4.07 mM, 4.08 mM, 4.09 mM, 4.1 mM, 4.11 mM, 4.12 mM, 4.13 mM, 4.14 mM, 4.15 mM, 4.16 mM, 4.17 mM, 4.18 mM, 4.19 mM, 4.2 mM, 4.21 mM, 4.22 mM, 4.23 mM, 4.24 mM, 4.25 mM, 4.26 mM, 4.27 mM, 4.28 mM, 4.29 mM, 4.3 mM, 4.31 mM, 4.32 mM, 4.33 mM, 4.34 mM, 4.35 mM, 4.36 mM, 4.37 mM, 4.38 mM, 4.39 mM, 4.4 mM, 4.41 mM, 4.42 mM, 4.43 mM, 4.44 mM, 4.45 mM, 4.46 mM, 4.47 mM, 4.48 mM, 4.49 mM, 4.5 mM, 4.51 mM, 4.52 mM, 4.53 mM, 4.54 mM, 4.55 mM, 4.56 mM, 4.57 mM, 4.58 mM, 4.59 mM, 4.6 mM, 4.61 mM, 4.62 mM, 4.63 mM, 4.64 mM, 4.65 mM, 4.66 mM, 4.67 mM, 4.68 mM, 4.69 mM, 4.7 mM, 4.71 mM, 4.72 mM, 4.73 mM, 4.74 mM, 4.75 mM, 4.76 mM, 4.77 mM, 4.78 mM, 4.79 mM, 4.8 mM, 4.81 mM, 4.82 mM, 4.83 mM, 4.84 mM, 4.85 mM, 4.86 mM, 4.87 mM, 4.88 mM, 4.89 mM, 4.9 mM, 4.91 mM, 4.92 mM, 4.93 mM, 4.94 mM, 4.95 mM, 4.96 mM, 4.97 mM, 4.98 mM, 4.99 mM, or about 5 mM, or any concentration therebetween.

In some embodiments, the reaction mixture may comprise a tRNA. In some embodiments, the tRNA may comprise an endogenous tRNA, i.e., a tRNA that is present in the cellular extract and derived from the cell. In some embodiments, the tRNA may comprise an exogenous tRNA, i.e., a tRNA that is added to the cellular extract. In some embodiments, the tRNA may be present in the reaction mixture at a concentration between about 10 μg/mL and about 1 mg/mL, e.g., about 10 μg/mL, 11 μg/mL, 12 μg/mL, 13 μg/mL, 14 μg/mL, 15 μg/mL, 16 μg/mL, 17 μg/mL, 18 μg/mL, 19 μg/mL, 20 μg/mL, 21 μg/mL, 22 μg/mL, 23 μg/mL, 24 μg/mL, 25 μg/mL, 26 μg/mL, 27 μg/mL, 28 μg/mL, 29 μg/mL, 30 μg/mL, 31 μg/mL, 32 μg/mL, 33 μg/mL, 34 μg/mL, 35 μg/mL, 36 μg/mL, 37 μg/mL, 38 μg/mL, 39 μg/mL, 40 μg/mL, 41 μg/mL, 42 μg/mL, 43 μg/mL, 44 μg/mL, 45 μg/mL, 46 μg/mL, 47 μg/mL, 48 μg/mL, 49 μg/mL, 50 μg/mL, 51 μg/mL, 52 μg/mL, 53 μg/mL, 54 μg/mL, 55 μg/mL, 56 μg/mL, 57 μg/mL, 58 μg/mL, 59 μg/mL, 60 μg/mL, 61 μg/mL, 62 μg/mL, 63 μg/mL, 64 μg/mL, 65 μg/mL, 66 μg/mL, 67 μg/mL, 68 μg/mL, 69 μg/mL, 70 μg/mL, 71 μg/mL, 72 μg/mL, 73 μg/mL, 74 μg/mL, 75 μg/mL, 76 μg/mL, 77 μg/mL, 78 μg/mL, 79 μg/mL, 80 μg/mL, 81 μg/mL, 82 μg/mL, 83 μg/mL, 84 μg/mL, 85 μg/mL, 86 μg/mL, 87 μg/mL, 88 μg/mL, 89 μg/mL, 90 μg/mL, 91 μg/mL, 92 μg/mL, 93 μg/mL, 94 μg/mL, 95 μg/mL, 96 μg/mL, 97 μg/mL, 98 μg/mL, 99 μg/mL, 100 μg/mL, 110 μg/mL, 120 μg/mL, 130 μg/mL, 140 μg/mL, 150 μg/mL, 160 μg/mL, 170 μg/mL, 180 μg/mL, 190 μg/mL, 200 μg/mL, 210 μg/mL, 220 μg/mL, 230 μg/mL, 240 μg/mL, 250 μg/mL, 260 μg/mL, 270 μg/mL, 280 μg/mL, 290 μg/mL, 300 μg/mL, 310 μg/mL, 320 μg/mL, 330 μg/mL, 340 μg/mL, 350 μg/mL, 360 μg/mL, 370 μg/mL, 380 μg/mL, 390 μg/mL, 400 μg/mL, 410 μg/mL, 420 μg/mL, 430 μg/mL, 440 μg/mL, 450 μg/mL, 460 μg/mL, 470 μg/mL, 480 μg/mL, 490 μg/mL, 500 μg/mL, 510 μg/mL, 520 μg/mL, 530 μg/mL, 540 μg/mL, 550 μg/mL, 560 μg/mL, 570 μg/mL, 580 μg/mL, 590 μg/mL, 600 μg/mL, 610 μg/mL, 620 μg/mL, 630 μg/mL, 640 μg/mL, 650 μg/mL, 660 μg/mL, 670 μg/mL, 680 μg/mL, 690 μg/mL, 700 μg/mL, 710 μg/mL, 720 μg/mL, 730 μg/mL, 740 μg/mL, 750 μg/mL, 760 μg/mL, 770 μg/mL, 780 μg/mL, 790 μg/mL, 800 μg/mL, 810 μg/mL, 820 μg/mL, 830 μg/mL, 840 μg/mL, 850 μg/mL, 860 μg/mL, 870 μg/mL, 880 μg/mL, 890 μg/mL, 900 μg/mL, 910 μg/mL, 920 μg/mL, 930 μg/mL, 940 μg/mL, 950 μg/mL, 960 μg/mL, 970 μg/mL, 980 μg/mL, 990 μg/mL, or about 1.0 mg/mL, or any concentration therebetween.

In some embodiments, the reaction mixture may comprise a chemical that modulates the oxidation-reduction potential of the cellular extract. In some embodiments, the chemical may comprise oxidized glutathione. In some embodiments, the chemical may comprise reduced glutathione. In some embodiments, the chemical may comprise a sulfhydryl inactivating agent as a pre-treatment of the extract prior to the start of the CFPS reaction. In some embodiments, the sulfhydryl inactivating agent may comprise iodoacetamide.

In some embodiments, the chemical may comprise oxidized or reduced glutathione at a concentration between about 0.1 mM and about 30 mM, e.g., about 0.1 mM, 0.11 mM, 0.12 mM, 0.13 mM, 0.14 mM, 0.15 mM, 0.16 mM, 0.17 mM, 0.18 mM, 0.19 mM, 0.2 mM, 0.21 mM, 0.22 mM, 0.23 mM, 0.24 mM, 0.25 mM, 0.26 mM, 0.27 mM, 0.28 mM, 0.29 mM, 0.3 mM, 0.31 mM, 0.32 mM, 0.33 mM, 0.34 mM, 0.35 mM, 0.36 mM, 0.37 mM, 0.38 mM, 0.39 mM, 0.4 mM, 0.41 mM, 0.42 mM, 0.43 mM, 0.44 mM, 0.45 mM, 0.46 mM, 0.47 mM, 0.48 mM, 0.49 mM, 0.5 mM, 0.51 mM, 0.52 mM, 0.53 mM, 0.54 mM, 0.55 mM, 0.56 mM, 0.57 mM, 0.58 mM, 0.59 mM, 0.6 mM, 0.61 mM, 0.62 mM, 0.63 mM, 0.64 mM, 0.65 mM, 0.66 mM, 0.67 mM, 0.68 mM, 0.69 mM, 0.7 mM, 0.71 mM, 0.72 mM, 0.73 mM, 0.74 mM, 0.75 mM, 0.76 mM, 0.77 mM, 0.78 mM, 0.79 mM, 0.8 mM, 0.81 mM, 0.82 mM, 0.83 mM, 0.84 mM, 0.85 mM, 0.86 mM, 0.87 mM, 0.88 mM, 0.89 mM, 0.9 mM, 0.91 mM, 0.92 mM, 0.93 mM, 0.94 mM, 0.95 mM, 0.96 mM, 0.97 mM, 0.98 mM, 0.99 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, 20 mM, 20.1 mM, 20.2 mM, 20.3 mM, 20.4 mM, 20.5 mM, 20.6 mM, 20.7 mM, 20.8 mM, 20.9 mM, 21 mM, 21.1 mM, 21.2 mM, 21.3 mM, 21.4 mM, 21.5 mM, 21.6 mM, 21.7 mM, 21.8 mM, 21.9 mM, 22 mM, 22.1 mM, 22.2 mM, 22.3 mM, 22.4 mM, 22.5 mM, 22.6 mM, 22.7 mM, 22.8 mM, 22.9 mM, 23 mM, 23.1 mM, 23.2 mM, 23.3 mM, 23.4 mM, 23.5 mM, 23.6 mM, 23.7 mM, 23.8 mM, 23.9 mM, 24 mM, 24.1 mM, 24.2 mM, 24.3 mM, 24.4 mM, 24.5 mM, 24.6 mM, 24.7 mM, 24.8 mM, 24.9 mM, 25 mM, 25.1 mM, 25.2 mM, 25.3 mM, 25.4 mM, 25.5 mM, 25.6 mM, 25.7 mM, 25.8 mM, 25.9 mM, 26 mM, 26.1 mM, 26.2 mM, 26.3 mM, 26.4 mM, 26.5 mM, 26.6 mM, 26.7 mM, 26.8 mM, 26.9 mM, 27 mM, 27.1 mM, 27.2 mM, 27.3 mM, 27.4 mM, 27.5 mM, 27.6 mM, 27.7 mM, 27.8 mM, 27.9 mM, 28 mM, 28.1 mM, 28.2 mM, 28.3 mM, 28.4 mM, 28.5 mM, 28.6 mM, 28.7 mM, 28.8 mM, 28.9 mM, 29 mM, 29.1 mM, 29.2 mM, 29.3 mM, 29.4 mM, 29.5 mM, 29.6 mM, 29.7 mM, 29.8 mM, 29.9 mM, or about 30 mM, or any concentration therebetween.

In some embodiments, the chemical may comprise a sulfhydryl inactivating agent at a concentration between about 1 μM and about 5000 μM, e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, 110 μM, 120 μM, 130 μM, 140 μM, 150 μM, 160 μM, 170 μM, 180 μM, 190 μM, 200 μM, 210 μM, 220 μM, 230 μM, 240 μM, 250 μM, 260 μM, 270 μM, 280 μM, 290 μM, 300 μM, 310 μM, 320 μM, 330 μM, 340 μM, 350 μM, 360 μM, 370 μM, 380 μM, 390 μM, 400 μM, 410 μM, 420 μM, 430 μM, 440 μM, 450 μM, 460 μM, 470 μM, 480 μM, 490 μM, 500 μM, 510 μM, 520 μM, 530 μM, 540 μM, 550 μM, 560 μM, 570 μM, 580 μM, 590 μM, 600 μM, 610 μM, 620 μM, 630 μM, 640 μM, 650 μM, 660 μM, 670 μM, 680 μM, 690 μM, 700 μM, 710 μM, 720 μM, 730 μM, 740 μM, 750 μM, 760 μM, 770 μM, 780 μM, 790 μM, 800 μM, 810 μM, 820 μM, 830 μM, 840 μM, 850 μM, 860 μM, 870 μM, 880 μM, 890 μM, 900 μM, 910 μM, 920 μM, 930 μM, 940 μM, 950 μM, 960 μM, 970 μM, 980 μM, 990 μM, 1000 μM, 1020 μM, 1040 μM, 1060 μM, 1080 μM, 1100 μM, 1120 μM, 1140 μM, 1160 μM, 1180 μM, 1200 μM, 1220 μM, 1240 μM, 1260 μM, 1280 μM, 1300 μM, 1320 μM, 1340 μM, 1360 μM, 1380 μM, 1400 μM, 1420 μM, 1440 μM, 1460 μM, 1480 μM, 1500 μM, 1520 μM, 1540 μM, 1560 μM, 1580 μM, 1600 μM, 1620 μM, 1640 μM, 1660 μM, 1680 μM, 1700 μM, 1720 μM, 1740 μM, 1760 μM, 1780 μM, 1800 μM, 1820 μM, 1840 μM, 1860 μM, 1880 μM, 1900 μM, 1920 μM, 1940 μM, 1960 μM, 1980 μM, 2000 μM, 2020 μM, 2040 μM, 2060 μM, 2080 μM, 2100 μM, 2120 μM, 2140 μM, 2160 μM, 2180 μM, 2200 μM, 2220 μM, 2240 μM, 2260 μM, 2280 μM, 2300 μM, 2320 μM, 2340 μM, 2360 μM, 2380 μM, 2400 μM, 2420 μM, 2440 μM, 2460 μM, 2480 μM, 2500 μM, 2520 μM, 2540 μM, 2560 μM, 2580 μM, 2600 μM, 2620 μM, 2640 μM, 2660 μM, 2680 μM, 2700 μM, 2720 μM, 2740 μM, 2760 μM, 2780 μM, 2800 μM, 2820 μM, 2840 μM, 2860 μM, 2880 μM, 2900 μM, 2920 μM, 2940 μM, 2960 μM, 2980 μM, 3000 μM, 3020 μM, 3040 μM, 3060 μM, 3080 μM, 3100 μM, 3120 μM, 3140 μM, 3160 μM, 3180 μM, 3200 μM, 3220 μM, 3240 μM, 3260 μM, 3280 μM, 3300 μM, 3320 μM, 3340 μM, 3360 μM, 3380 μM, 3400 μM, 3420 μM, 3440 μM, 3460 μM, 3480 μM, 3500 μM, 3520 μM, 3540 μM, 3560 μM, 3580 μM, 3600 μM, 3620 μM, 3640 μM, 3660 μM, 3680 μM, 3700 μM, 3720 μM, 3740 μM, 3760 μM, 3780 μM, 3800 μM, 3820 μM, 3840 μM, 3860 μM, 3880 μM, 3900 μM, 3920 μM, 3940 μM, 3960 μM, 3980 μM, 4000 μM, 4020 μM, 4040 μM, 4060 μM, 4080 μM, 4100 μM, 4120 μM, 4140 μM, 4160 μM, 4180 μM, 4200 μM, 4220 μM, 4240 μM, 4260 μM, 4280 μM, 4300 μM, 4320 μM, 4340 μM, 4360 μM, 4380 μM, 4400 μM, 4420 μM, 4440 μM, 4460 μM, 4480 μM, 4500 μM, 4520 μM, 4540 μM, 4560 μM, 4580 μM, 4600 μM, 4620 μM, 4640 μM, 4660 μM, 4680 μM, 4700 μM, 4720 μM, 4740 μM, 4760 μM, 4780 μM, 4800 μM, 4820 μM, 4840 μM, 4860 μM, 4880 μM, 4900 μM, 4920 μM, 4940 μM, 4960 μM, 4980 μM, or about 5000 μM, or any concentration therebetween.

In some embodiments, the reaction mixture may comprise at least one chemical that operates to facilitate the proper folding and/or post-translational modification of the polypeptide or protein being produced. In some embodiments the chemical comprises a protein. In some embodiments, the chemical comprises a chaperone. In some embodiments, the chemical comprises an enzyme. In some embodiments, the chemical comprises a glycosyltransferase. In some embodiments, the chemical comprises a sugar donor. In some embodiments, the polypeptide or protein being produced may be expressed from a plasmid or the genome of the cell used to produce the cellular extract.

In some embodiments, the reaction mixture may comprise an alcohol. In some embodiments, the alcohol may comprise a polyol. In some embodiments, the alcohol may be present at a concentration between about 0% (v/v) and about 25% (v/v), e.g., about 0.1% (v/v), 0.2% (v/v), 0.3% (v/v), 0.4% (v/v), 0.5% (v/v), 0.6% (v/v), 0.7% (v/v), 0.8% (v/v), 0.9% (v/v), 1% (v/v), 1.1% (v/v), 1.2% (v/v), 1.3% (v/v), 1.4% (v/v), 1.5% (v/v), 1.6% (v/v), 1.7% (v/v), 1.8% (v/v), 1.9% (v/v), 2% (v/v), 2.1% (v/v), 2.2% (v/v), 2.3% (v/v), 2.4% (v/v), 2.5% (v/v), 2.6% (v/v), 2.7% (v/v), 2.8% (v/v), 2.9% (v/v), 3% (v/v), 3.1% (v/v), 3.2% (v/v), 3.3% (v/v), 3.4% (v/v), 3.5% (v/v), 3.6% (v/v), 3.7% (v/v), 3.8% (v/v), 3.9% (v/v), 4% (v/v), 4.1% (v/v), 4.2% (v/v), 4.3% (v/v), 4.4% (v/v), 4.5% (v/v), 4.6% (v/v), 4.7% (v/v), 4.8% (v/v), 4.9% (v/v), 5% (v/v), 5.1% (v/v), 5.2% (v/v), 5.3% (v/v), 5.4% (v/v), 5.5% (v/v), 5.6% (v/v), 5.7% (v/v), 5.8% (v/v), 5.9% (v/v), 6% (v/v), 6.1% (v/v), 6.2% (v/v), 6.3% (v/v), 6.4% (v/v), 6.5% (v/v), 6.6% (v/v), 6.7% (v/v), 6.8% (v/v), 6.9% (v/v), 7% (v/v), 7.1% (v/v), 7.2% (v/v), 7.3% (v/v), 7.4% (v/v), 7.5% (v/v), 7.6% (v/v), 7.7% (v/v), 7.8% (v/v), 7.9% (v/v), 8% (v/v), 8.1% (v/v), 8.2% (v/v), 8.3% (v/v), 8.4% (v/v), 8.5% (v/v), 8.6% (v/v), 8.7% (v/v), 8.8% (v/v), 8.9% (v/v), 9% (v/v), 9.1% (v/v), 9.2% (v/v), 9.3% (v/v), 9.4% (v/v), 9.5% (v/v), 9.6% (v/v), 9.7% (v/v), 9.8% (v/v), 9.9% (v/v), 10% (v/v), 10.1% (v/v), 10.2% (v/v), 10.3% (v/v), 10.4% (v/v), 10.5% (v/v), 10.6% (v/v), 10.7% (v/v), 10.8% (v/v), 10.9% (v/v), 11% (v/v), 11.1% (v/v), 11.2% (v/v), 11.3% (v/v), 11.4% (v/v), 11.5% (v/v), 11.6% (v/v), 11.7% (v/v), 11.8% (v/v), 11.9% (v/v), 12% (v/v), 12.1% (v/v), 12.2% (v/v), 12.3% (v/v), 12.4% (v/v), 12.5% (v/v), 12.6% (v/v), 12.7% (v/v), 12.8% (v/v), 12.9% (v/v), 13% (v/v), 13.1% (v/v), 13.2% (v/v), 13.3% (v/v), 13.4% (v/v), 13.5% (v/v), 13.6% (v/v), 13.7% (v/v), 13.8% (v/v), 13.9% (v/v), 14% (v/v), 14.1% (v/v), 14.2% (v/v), 14.3% (v/v), 14.4% (v/v), 14.5% (v/v), 14.6% (v/v), 14.7% (v/v), 14.8% (v/v), 14.9% (v/v), 15% (v/v), 15.1% (v/v), 15.2% (v/v), 15.3% (v/v), 15.4% (v/v), 15.5% (v/v), 15.6% (v/v), 15.7% (v/v), 15.8% (v/v), 15.9% (v/v), 16% (v/v), 16.1% (v/v), 16.2% (v/v), 16.3% (v/v), 16.4% (v/v), 16.5% (v/v), 16.6% (v/v), 16.7% (v/v), 16.8% (v/v), 16.9% (v/v), 17% (v/v), 17.1% (v/v), 17.2% (v/v), 17.3% (v/v), 17.4% (v/v), 17.5% (v/v), 17.6% (v/v), 17.7% (v/v), 17.8% (v/v), 17.9% (v/v), 18% (v/v), 18.1% (v/v), 18.2% (v/v), 18.3% (v/v), 18.4% (v/v), 18.5% (v/v), 18.6% (v/v), 18.7% (v/v), 18.8% (v/v), 18.9% (v/v), 19% (v/v), 19.1% (v/v), 19.2% (v/v), 19.3% (v/v), 19.4% (v/v), 19.5% (v/v), 19.6% (v/v), 19.7% (v/v), 19.8% (v/v), 19.9% (v/v), 20% (v/v), 20.1% (v/v), 20.2% (v/v), 20.3% (v/v), 20.4% (v/v), 20.5% (v/v), 20.6% (v/v), 20.7% (v/v), 20.8% (v/v), 20.9% (v/v), 21% (v/v), 21.1% (v/v), 21.2% (v/v), 21.3% (v/v), 21.4% (v/v), 21.5% (v/v), 21.6% (v/v), 21.7% (v/v), 21.8% (v/v), 21.9% (v/v), 22% (v/v), 22.1% (v/v), 22.2% (v/v), 22.3% (v/v), 22.4% (v/v), 22.5% (v/v), 22.6% (v/v), 22.7% (v/v), 22.8% (v/v), 22.9% (v/v), 23% (v/v), 23.1% (v/v), 23.2% (v/v), 23.3% (v/v), 23.4% (v/v), 23.5% (v/v), 23.6% (v/v), 23.7% (v/v), 23.8% (v/v), 23.9% (v/v), 24% (v/v), 24.1% (v/v), 24.2% (v/v), 24.3% (v/v), 24.4% (v/v), 24.5% (v/v), 24.6% (v/v), 24.7% (v/v), 24.8% (v/v), 24.9% (v/v), or about 25% (v/v), or any concentration therebetween.

In some embodiments, the energy source in the reaction mixture may be present at a concentration between about 10 mM and about 400 mM, e.g., about 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, 220 mM, 221 mM, 222 mM, 223 mM, 224 mM, 225 mM, 226 mM, 227 mM, 228 mM, 229 mM, 230 mM, 231 mM, 232 mM, 233 mM, 234 mM, 235 mM, 236 mM, 237 mM, 238 mM, 239 mM, 240 mM, 241 mM, 242 mM, 243 mM, 244 mM, 245 mM, 246 mM, 247 mM, 248 mM, 249 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 287 mM, 288 mM, 289 mM, 290 mM, 291 mM, 292 mM, 293 mM, 294 mM, 295 mM, 296 mM, 297 mM, 298 mM, 299 mM, 300 mM, 301 mM, 302 mM, 303 mM, 304 mM, 305 mM, 306 mM, 307 mM, 308 mM, 309 mM, 310 mM, 311 mM, 312 mM, 313 mM, 314 mM, 315 mM, 316 mM, 317 mM, 318 mM, 319 mM, 320 mM, 321 mM, 322 mM, 323 mM, 324 mM, 325 mM, 326 mM, 327 mM, 328 mM, 329 mM, 330 mM, 331 mM, 332 mM, 333 mM, 334 mM, 335 mM, 336 mM, 337 mM, 338 mM, 339 mM, 340 mM, 341 mM, 342 mM, 343 mM, 344 mM, 345 mM, 346 mM, 347 mM, 348 mM, 349 mM, 350 mM, 351 mM, 352 mM, 353 mM, 354 mM, 355 mM, 356 mM, 357 mM, 358 mM, 359 mM, 360 mM, 361 mM, 362 mM, 363 mM, 364 mM, 365 mM, 366 mM, 367 mM, 368 mM, 369 mM, 370 mM, 371 mM, 372 mM, 373 mM, 374 mM, 375 mM, 376 mM, 377 mM, 378 mM, 379 mM, 380 mM, 381 mM, 382 mM, 383 mM, 384 mM, 385 mM, 386 mM, 387 mM, 388 mM, 389 mM, 390 mM, 391 mM, 392 mM, 393 mM, 394 mM, 395 mM, 396 mM, 397 mM, 398 mM, 399 mM, or about 400 mM, or any concentration therebetween. In some embodiments, the concentration listed above may be applicable to any energy source disclosed herein, including but not limited to, a phosphate group, a non-phosphorylated energy group, or one or more selected from phosphoenolpyruvate, glutamate, glycerol, pyruvate, glucose, and creatine phosphate.

In some embodiments, the salt in the reaction mixture may comprise potassium at a concentration between about 50 mM and about 500 mM, e.g., about 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 201 mM, 202 mM, 203 mM, 204 mM, 205 mM, 206 mM, 207 mM, 208 mM, 209 mM, 210 mM, 211 mM, 212 mM, 213 mM, 214 mM, 215 mM, 216 mM, 217 mM, 218 mM, 219 mM, 220 mM, 221 mM, 222 mM, 223 mM, 224 mM, 225 mM, 226 mM, 227 mM, 228 mM, 229 mM, 230 mM, 231 mM, 232 mM, 233 mM, 234 mM, 235 mM, 236 mM, 237 mM, 238 mM, 239 mM, 240 mM, 241 mM, 242 mM, 243 mM, 244 mM, 245 mM, 246 mM, 247 mM, 248 mM, 249 mM, 250 mM, 251 mM, 252 mM, 253 mM, 254 mM, 255 mM, 256 mM, 257 mM, 258 mM, 259 mM, 260 mM, 261 mM, 262 mM, 263 mM, 264 mM, 265 mM, 266 mM, 267 mM, 268 mM, 269 mM, 270 mM, 271 mM, 272 mM, 273 mM, 274 mM, 275 mM, 276 mM, 277 mM, 278 mM, 279 mM, 280 mM, 281 mM, 282 mM, 283 mM, 284 mM, 285 mM, 286 mM, 287 mM, 288 mM, 289 mM, 290 mM, 291 mM, 292 mM, 293 mM, 294 mM, 295 mM, 296 mM, 297 mM, 298 mM, 299 mM, 300 mM, 301 mM, 302 mM, 303 mM, 304 mM, 305 mM, 306 mM, 307 mM, 308 mM, 309 mM, 310 mM, 311 mM, 312 mM, 313 mM, 314 mM, 315 mM, 316 mM, 317 mM, 318 mM, 319 mM, 320 mM, 321 mM, 322 mM, 323 mM, 324 mM, 325 mM, 326 mM, 327 mM, 328 mM, 329 mM, 330 mM, 331 mM, 332 mM, 333 mM, 334 mM, 335 mM, 336 mM, 337 mM, 338 mM, 339 mM, 340 mM, 341 mM, 342 mM, 343 mM, 344 mM, 345 mM, 346 mM, 347 mM, 348 mM, 349 mM, 350 mM, 351 mM, 352 mM, 353 mM, 354 mM, 355 mM, 356 mM, 357 mM, 358 mM, 359 mM, 360 mM, 361 mM, 362 mM, 363 mM, 364 mM, 365 mM, 366 mM, 367 mM, 368 mM, 369 mM, 370 mM, 371 mM, 372 mM, 373 mM, 374 mM, 375 mM, 376 mM, 377 mM, 378 mM, 379 mM, 380 mM, 381 mM, 382 mM, 383 mM, 384 mM, 385 mM, 386 mM, 387 mM, 388 mM, 389 mM, 390 mM, 391 mM, 392 mM, 393 mM, 394 mM, 395 mM, 396 mM, 397 mM, 398 mM, 399 mM, 400 mM, 401 mM, 402 mM, 403 mM, 404 mM, 405 mM, 406 mM, 407 mM, 408 mM, 409 mM, 410 mM, 411 mM, 412 mM, 413 mM, 414 mM, 415 mM, 416 mM, 417 mM, 418 mM, 419 mM, 420 mM, 421 mM, 422 mM, 423 mM, 424 mM, 425 mM, 426 mM, 427 mM, 428 mM, 429 mM, 430 mM, 431 mM, 432 mM, 433 mM, 434 mM, 435 mM, 436 mM, 437 mM, 438 mM, 439 mM, 440 mM, 441 mM, 442 mM, 443 mM, 444 mM, 445 mM, 446 mM, 447 mM, 448 mM, 449 mM, 450 mM, 451 mM, 452 mM, 453 mM, 454 mM, 455 mM, 456 mM, 457 mM, 458 mM, 459 mM, 460 mM, 461 mM, 462 mM, 463 mM, 464 mM, 465 mM, 466 mM, 467 mM, 468 mM, 469 mM, 470 mM, 471 mM, 472 mM, 473 mM, 474 mM, 475 mM, 476 mM, 477 mM, 478 mM, 479 mM, 480 mM, 481 mM, 482 mM, 483 mM, 484 mM, 485 mM, 486 mM, 487 mM, 488 mM, 489 mM, 490 mM, 491 mM, 492 mM, 493 mM, 494 mM, 495 mM, 496 mM, 497 mM, 498 mM, 499 mM, or about 500 mM, or any concentration therebetween.

In some embodiments, the salt in the reaction mixture may comprise magnesium at a concentration between about 1 mM and about 30 mM, e.g., about 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, 20 mM, 20.1 mM, 20.2 mM, 20.3 mM, 20.4 mM, 20.5 mM, 20.6 mM, 20.7 mM, 20.8 mM, 20.9 mM, 21 mM, 21.1 mM, 21.2 mM, 21.3 mM, 21.4 mM, 21.5 mM, 21.6 mM, 21.7 mM, 21.8 mM, 21.9 mM, 22 mM, 22.1 mM, 22.2 mM, 22.3 mM, 22.4 mM, 22.5 mM, 22.6 mM, 22.7 mM, 22.8 mM, 22.9 mM, 23 mM, 23.1 mM, 23.2 mM, 23.3 mM, 23.4 mM, 23.5 mM, 23.6 mM, 23.7 mM, 23.8 mM, 23.9 mM, 24 mM, 24.1 mM, 24.2 mM, 24.3 mM, 24.4 mM, 24.5 mM, 24.6 mM, 24.7 mM, 24.8 mM, 24.9 mM, 25 mM, 25.1 mM, 25.2 mM, 25.3 mM, 25.4 mM, 25.5 mM, 25.6 mM, 25.7 mM, 25.8 mM, 25.9 mM, 26 mM, 26.1 mM, 26.2 mM, 26.3 mM, 26.4 mM, 26.5 mM, 26.6 mM, 26.7 mM, 26.8 mM, 26.9 mM, 27 mM, 27.1 mM, 27.2 mM, 27.3 mM, 27.4 mM, 27.5 mM, 27.6 mM, 27.7 mM, 27.8 mM, 27.9 mM, 28 mM, 28.1 mM, 28.2 mM, 28.3 mM, 28.4 mM, 28.5 mM, 28.6 mM, 28.7 mM, 28.8 mM, 28.9 mM, 29 mM, 29.1 mM, 29.2 mM, 29.3 mM, 29.4 mM, 29.5 mM, 29.6 mM, 29.7 mM, 29.8 mM, 29.9 mM, or 30 mM, or any concentration therebetween. In some embodiments, the salt in the reaction mixture may comprise potassium at a concentration disclosed above and magnesium at a concentration disclosed above.

In some embodiments, the salt in the reaction mixture may comprise ammonium at a concentration between about 1 mM and about 400 mM, e.g., about 1 mM, 1.1 mM, 1.2 mM, 1.3 mM, 1.4 mM, 1.5 mM, 1.6 mM, 1.7 mM, 1.8 mM, 1.9 mM, 2 mM, 2.1 mM, 2.2 mM, 2.3 mM, 2.4 mM, 2.5 mM, 2.6 mM, 2.7 mM, 2.8 mM, 2.9 mM, 3 mM, 3.1 mM, 3.2 mM, 3.3 mM, 3.4 mM, 3.5 mM, 3.6 mM, 3.7 mM, 3.8 mM, 3.9 mM, 4 mM, 4.1 mM, 4.2 mM, 4.3 mM, 4.4 mM, 4.5 mM, 4.6 mM, 4.7 mM, 4.8 mM, 4.9 mM, 5 mM, 5.1 mM, 5.2 mM, 5.3 mM, 5.4 mM, 5.5 mM, 5.6 mM, 5.7 mM, 5.8 mM, 5.9 mM, 6 mM, 6.1 mM, 6.2 mM, 6.3 mM, 6.4 mM, 6.5 mM, 6.6 mM, 6.7 mM, 6.8 mM, 6.9 mM, 7 mM, 7.1 mM, 7.2 mM, 7.3 mM, 7.4 mM, 7.5 mM, 7.6 mM, 7.7 mM, 7.8 mM, 7.9 mM, 8 mM, 8.1 mM, 8.2 mM, 8.3 mM, 8.4 mM, 8.5 mM, 8.6 mM, 8.7 mM, 8.8 mM, 8.9 mM, 9 mM, 9.1 mM, 9.2 mM, 9.3 mM, 9.4 mM, 9.5 mM, 9.6 mM, 9.7 mM, 9.8 mM, 9.9 mM, 10 mM, 10.1 mM, 10.2 mM, 10.3 mM, 10.4 mM, 10.5 mM, 10.6 mM, 10.7 mM, 10.8 mM, 10.9 mM, 11 mM, 11.1 mM, 11.2 mM, 11.3 mM, 11.4 mM, 11.5 mM, 11.6 mM, 11.7 mM, 11.8 mM, 11.9 mM, 12 mM, 12.1 mM, 12.2 mM, 12.3 mM, 12.4 mM, 12.5 mM, 12.6 mM, 12.7 mM, 12.8 mM, 12.9 mM, 13 mM, 13.1 mM, 13.2 mM, 13.3 mM, 13.4 mM, 13.5 mM, 13.6 mM, 13.7 mM, 13.8 mM, 13.9 mM, 14 mM, 14.1 mM, 14.2 mM, 14.3 mM, 14.4 mM, 14.5 mM, 14.6 mM, 14.7 mM, 14.8 mM, 14.9 mM, 15 mM, 15.1 mM, 15.2 mM, 15.3 mM, 15.4 mM, 15.5 mM, 15.6 mM, 15.7 mM, 15.8 mM, 15.9 mM, 16 mM, 16.1 mM, 16.2 mM, 16.3 mM, 16.4 mM, 16.5 mM, 16.6 mM, 16.7 mM, 16.8 mM, 16.9 mM, 17 mM, 17.1 mM, 17.2 mM, 17.3 mM, 17.4 mM, 17.5 mM, 17.6 mM, 17.7 mM, 17.8 mM, 17.9 mM, 18 mM, 18.1 mM, 18.2 mM, 18.3 mM, 18.4 mM, 18.5 mM, 18.6 mM, 18.7 mM, 18.8 mM, 18.9 mM, 19 mM, 19.1 mM, 19.2 mM, 19.3 mM, 19.4 mM, 19.5 mM, 19.6 mM, 19.7 mM, 19.8 mM, 19.9 mM, 20 mM, 20.5 mM, 21 mM, 21.5 mM, 22 mM, 22.5 mM, 23 mM, 23.5 mM, 24 mM, 24.5 mM, 25 mM, 25.5 mM, 26 mM, 26.5 mM, 27 mM, 27.5 mM, 28 mM, 28.5 mM, 29 mM, 29.5 mM, 30 mM, 30.5 mM, 31 mM, 31.5 mM, 32 mM, 32.5 mM, 33 mM, 33.5 mM, 34 mM, 34.5 mM, 35 mM, 35.5 mM, 36 mM, 36.5 mM, 37 mM, 37.5 mM, 38 mM, 38.5 mM, 39 mM, 39.5 mM, 40 mM, 40.5 mM, 41 mM, 41.5 mM, 42 mM, 42.5 mM, 43 mM, 43.5 mM, 44 mM, 44.5 mM, 45 mM, 45.5 mM, 46 mM, 46.5 mM, 47 mM, 47.5 mM, 48 mM, 48.5 mM, 49 mM, 49.5 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, 101 mM, 102 mM, 103 mM, 104 mM, 105 mM, 106 mM, 107 mM, 108 mM, 109 mM, 110 mM, 111 mM, 112 mM, 113 mM, 114 mM, 115 mM, 116 mM, 117 mM, 118 mM, 119 mM, 120 mM, 121 mM, 122 mM, 123 mM, 124 mM, 125 mM, 126 mM, 127 mM, 128 mM, 129 mM, 130 mM, 131 mM, 132 mM, 133 mM, 134 mM, 135 mM, 136 mM, 137 mM, 138 mM, 139 mM, 140 mM, 141 mM, 142 mM, 143 mM, 144 mM, 145 mM, 146 mM, 147 mM, 148 mM, 149 mM, 150 mM, 151 mM, 152 mM, 153 mM, 154 mM, 155 mM, 156 mM, 157 mM, 158 mM, 159 mM, 160 mM, 161 mM, 162 mM, 163 mM, 164 mM, 165 mM, 166 mM, 167 mM, 168 mM, 169 mM, 170 mM, 171 mM, 172 mM, 173 mM, 174 mM, 175 mM, 176 mM, 177 mM, 178 mM, 179 mM, 180 mM, 181 mM, 182 mM, 183 mM, 184 mM, 185 mM, 186 mM, 187 mM, 188 mM, 189 mM, 190 mM, 191 mM, 192 mM, 193 mM, 194 mM, 195 mM, 196 mM, 197 mM, 198 mM, 199 mM, 200 mM, 205 mM, 210 mM, 215 mM, 220 mM, 225 mM, 230 mM, 235 mM, 240 mM, 245 mM, 250 mM, 255 mM, 260 mM, 265 mM, 270 mM, 275 mM, 280 mM, 285 mM, 290 mM, 295 mM, 300 mM, 305 mM, 310 mM, 315 mM, 320 mM, 325 mM, 330 mM, 335 mM, 340 mM, 345 mM, 350 mM, 355 mM, 360 mM, 365 mM, 370 mM, 375 mM, 380 mM, 385 mM, 390 mM, 395 mM, or about 400 mM, or any concentration therebetween.

In some embodiments, the reaction mixture may comprise a nucleic acid transcription template. In some embodiments, the reaction mixture may comprise a nucleic acid translation template. In some embodiments, the reaction mixture may comprise both a nucleic acid transcription template and a nucleic acid translation template. In some embodiments, the reaction mixture may comprise a polymerase. In some embodiments, the polymerase may generate a nucleic acid translation template from a nucleic acid transcription template. In some embodiments, the polymerase may comprise a DNA-dependent RNA polymerase. In some embodiments, the polymerase may comprise an endogenous polymerase, i.e., a polymerase that is present in the cellular extract and derived from the cell. In some embodiments, the polymerase may comprise an exogenous polymerase, i.e., a polymerase that is added to the cellular extract. In some embodiments, the polymerase may comprise T7 RNA polymerase. In some embodiments, the polymerase may be expressed from a plasmid present in the cell used for preparation of the cellular extract. In some embodiments, the polymerase may be expressed from a site in the genome of the cell used for preparation of the cellular extract.

In some embodiments, the nucleic acid encoding the polypeptide may comprise a DNA. In some embodiments, the DNA may comprise an open reading frame of the polypeptide. The open reading frame is the amino acid coding sequence of a polypeptide. In some embodiments, the DNA may comprise a promoter for a DNA-dependent RNA polymerase. In some embodiments, the open reading frame of the polypeptide may be operatively linked to the promoter. In some embodiments, the DNA may comprise a ribosome binding site located at or upstream of the 5′ end of the open reading frame. In some embodiments, the DNA may comprise a terminator located at or downstream of the 3′ end of the open reading frame. In some embodiments, the promoter may comprise a T7 promoter. In some embodiments, the T7 promoter may comprise a sequence having at least 80%, 81%, 82%, 83%, 84%, 85%, 86%, 87%, 88%, 89%9, 90%, 91%, 92%, 93%, 94%, 95%, 96%, 97%, 98%, or at least 99%, sequence identity with 5′-TAATACGACTCACTATAGG-3′ (SEQ ID No: 1). In some embodiments, the T7 promoter may comprise SEQ ID No: 1. In some embodiments, the terminator may comprise a T7 terminator. In some embodiments, the T7 terminator may comprise a sequence having at least 80%, 81%, 82%, 83%, 84%, 85%, 86%, 87%, 88%, 89%, 90%, 91%, 92%, 93%, 94%, 95%, 96%, 97%, 98%, or at least 99%, sequence identity with 5′-CTAGCATAACCCCTTGGGGCCTCTAAACGGGTCTTGAGGGGTTTTTTG-3′ (SEQ ID No: 2). In some embodiments, the T7 terminator may comprise SEQ ID No: 2.

In some embodiments, the method may comprise a step of incubating the reaction mixture to produce the polypeptide. In some embodiments, the reaction mixture is not subject to clarification through centrifugation prior to the step of the incubating. In some embodiments, the method for CFPS disclosed herein may further comprise a step of isolating the polypeptide from the reaction mixture.

In some embodiments, the method disclosed herein may be carried out at a temperature between about 1° C. and about 40° C., e.g., about 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or about 40° C., or any temperature therebetween. In some embodiments, the temperatures disclosed above may be applicable to one or more steps of the method disclosed herein, including but not limited to, the steps of growing the cell, harvesting the cell, lysing the cell to produce the cellular lysate, passing the cellular lysate through the filter to produce the cellular extract, contacting the cellular extract with the nucleic acid encoding a polypeptide to produce the reaction mixture, incubating the reaction mixture to produce the polypeptide, and isolating the polypeptide from the reaction mixture.

In some embodiments, the step of incubating the reaction mixture to produce the polypeptide may be carried out at an appropriate temperature. In some embodiments, the step of incubating the reaction mixture to produce the polypeptide may be carried out at a temperature between about 10° C. and about 42° C., e.g., about 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., 40° C., 40.5° C., 41° C., 41.5° C., or about 42° C., or any temperature therebetween.

In some embodiments, the step of isolating the polypeptide from the reaction mixture may be carried out at a temperature between about 1° C. and about 30° C., e.g., about 1° C., 1.5° C., 2° C., 2.5° C., 3° C., 3.5° C., 4° C., 4.5° C., 5° C., 5.5° C., 6° C., 6.5° C., 7° C., 7.5° C., 8° C., 8.5° C., 9° C., 9.5° C., 10° C., 10.5° C., 11° C., 11.5° C., 12° C., 12.5° C., 13° C., 13.5° C., 14° C., 14.5° C., 15° C., 15.5° C., 16° C., 16.5° C., 17° C., 17.5° C., 18° C., 18.5° C., 19° C., 19.5° C., 20° C., 20.5° C., 21° C., 21.5° C., 22° C., 22.5° C., 23° C., 23.5° C., 24° C., 24.5° C., 25° C., 25.5° C., 26° C., 26.5° C., 27° C., 27.5° C., 28° C., 28.5° C., 29° C., 29.5° C., or 30° C., or any temperature therebetween.

In some embodiments, the method may be carried out at a temperature between about 1° C. and about 40° C. In some embodiments, the method may be carried out at a temperature between about 2° C. and about 39° C. In some embodiments, the method may be carried out at a temperature between about 3° C. and about 38° C. In some embodiments, the method may be carried out at a temperature between about 4° C. and about 37° C. In some embodiments, the method may be carried out at a temperature between about 5° C. and about 36° C. In some embodiments, the method may be carried out at a temperature between about 6° C. and about 35° C. In some embodiments, the method may be carried out at a temperature between about 7° C. and about 34° C. In some embodiments, the method may be carried out at a temperature between about 8° C. and about 33° C. In some embodiments, the method may be carried out at a temperature between about 9° C. and about 32° C. In some embodiments, the method may be carried out at a temperature between about 10° C. and about 31° C. In some embodiments, the method may be carried out at a temperature between about 11° C. and about 30° C. In some embodiments, the method may be carried out at a temperature between about 12° C. and about 29° C. In some embodiments, the method may be carried out at a temperature between about 13° C. and about 28° C. In some embodiments, the method may be carried out at a temperature between about 14° C. and about 27° C. In some embodiments, the method may be carried out at a temperature between about 15° C. and about 26° C. In some embodiments, the method may be carried out at a temperature between about 16° C. and about 25° C. In some embodiments, the method may be carried out at a temperature between about 17° C. and about 24° C. In some embodiments, the method may be carried out at a temperature between about 18° C. and about 23° C. In some embodiments, the method may be carried out at a temperature between about 19° C. and about 22° C. In some embodiments, the method may be carried out at a temperature between about 20° C. and about 21° C. In some embodiments, the temperatures disclosed above may be applicable to one or more steps of the method disclosed herein, including but not limited to, the steps of growing a cell, harvesting a cell, lysing the cell to produce the cellular lysate, passing the cellular lysate through the filter to produce the cellular extract, contacting the cellular extract with the nucleic acid encoding a polypeptide to produce the reaction mixture, and incubating the reaction mixture to produce the polypeptide.

### Characterization of Clarified Cellular Extract Via Filtration

The properties of a cellular extract clarified according to a method disclosed herein can be characterized. For example, the biological activity of the cellular extract can be characterized by a volumetric yield for batch reactions that produce a reporter protein, such as a green fluorescent protein (GFP) or luciferase.

In some embodiments, a cellular extract clarified according to a method disclosed herein may be part of a CFPS reaction mixture. A reaction mixture is complete if it contains all reagents necessary to perform the cell-free polypeptide synthesis reaction. Components of a reaction mixture may be stored separately in separate containers, and each contains one or more of the components. Components of a reaction mixture may be packaged separately for commercialization, and a commercial kit may contain one or more of the reaction components for a reaction mixture. A cellular extract clarified according to a method disclosed herein may be separately packaged as a component of a CFPS reaction mixture kit.

### Systems for Clarification of Cellular Lysate Via Filtration

Disclosed herein, in some embodiments, is a system for cell-free polypeptide synthesis (CFPS). In some embodiments, the system may be used for preparation of clarified cellular extract for CFPS. In some embodiments, the system may operate in a batch, continuous, or semi-continuous manner. In some embodiments, the system may be enclosed in a closed sterile environment. In some embodiments, the system may perform the entire clarification process under a sterile condition.

Disclosed herein, in some embodiments, is a system for CFPS. In some embodiments, the system may comprise one or more of the devices disclosed herein. In some embodiments, the system may comprise a filter disclosed herein. In some embodiments, the filter may receive a cellular lysate and clarify the cellular lysate through filtration to produce a cellular extract for CFPS. In some embodiments, the system may comprise a pump. In some embodiments, the pump may be configured to control the flow and/or pressure of the cellular lysate inside the system. In some embodiments, the filter may comprise at least one, two or more filters disclosed herein. For example, in some embodiments, the filter may comprise a medium comprising a pore configured to remove from the cellular lysate a particle with a size greater than about 0.4 micron, about 0.2 micron, or about 0.1 micron. In some embodiments, the filter may comprise a first filter and a second filter. In some embodiments, the first filter may comprise a depth filter. In some embodiments, the depth filter may comprise a lenticular, a deep bed, a pad, a panel, a cartridge-type depth filter, or a combination thereof. In some embodiments, the second filter may comprise a surface filter. In some embodiments, the second filter may comprise a depth filter. In some embodiments, the second filter may comprise a filter comprising a sterilization-grade membrane. In some embodiments, the system may comprise other devices disclosed herein, including but not limited to, a cell culture vessel, a cell harvesting device, a cell lysis device, a device for CFPS, or a polypeptide isolation device.

In some embodiments, the CFPS system may comprise a device used in a CFPS method disclosed herein. In some embodiments, the system may comprise a filter disclosed herein for clarification of a cellular lysate. In some embodiments, the filter may be configured to receive a cellular lysate. In some embodiments, the filter may comprise at least two filters disclosed herein. In some embodiments, the at least two filters may be in fluid communication with each other. In some embodiments, the filter may comprise a depth filter disclosed herein. In some embodiments, the system may comprise a filter disclosed herein that retains a particle with a size greater than about 0.5 micron. In some embodiments, the system may comprise a first filter disclosed herein that retains a particle with a size greater than about 15 microns. In some embodiments, the system may comprise a first filter disclosed herein that retains a particle with a size between about 0.5 micron and about 15 microns. In some embodiments, the system may comprise a second filter disclosed herein that retains a particle with a size greater than about 0.5 micron. In some embodiments, the system may comprise a second filter disclosed herein that retains a particle with a size between about 0.2 micron and about 0.5 micron. In some embodiments, the system may comprise a filter having a sterilization-grade membrane disclosed herein. In some embodiments, the filter having a sterilization-grade membrane may retain a particle with a size greater than about 0.1 micron. In some embodiments, the filter having a sterilization-grade membrane may retain a particle with a size greater than about 0.2 micron. In some embodiments, the filter having a sterilization-grade membrane may retain a particle with a size greater than about 0.4 micron.

In some embodiments, the system may further comprise a bioreactor. In some embodiments, the bioreactor may contain a cell disclosed herein that is used for preparation of cellular extract for CFPS. In some embodiments, the cell may comprise a cultured cell disclosed herein. In some embodiments, the cell may comprise at least two cells. In some embodiments, the cell may comprise a cell culture disclosed herein. In some embodiments, the bioreactor may comprise a batch bioreactor, a fed batch bioreactor, a continuous bioreactor, or a combination thereof. In some embodiments, the continuous bioreactor may comprise a continuous perfusion fermentation bioreactor. In some embodiments, the bioreactor may comprise any suitable material. In some embodiments, the material may comprise stainless steel, plastic, or a combination thereof. In some embodiments, the bioreactor may be of any suitable size. In some embodiments, the bioreactor may comprise a single-use bioreactor. In some embodiments, the single-use bioreactor may be in the form of a flexible, collapsible bag.

In some embodiments, the system may comprise a cell lysing instrument disclosed herein. In some embodiments, a cell lysing process disclosed herein may be carried out in the bioreactor. In some embodiments, the system may comprise a shear or sonication device used to disrupt the cell membrane to produce the cellular lysate. In some embodiments, the bioreactor may be in fluid communication with another component of the system. In some embodiments, a fluid may be recirculated from the bioreactor to a cell lysing device. In some embodiments, a fluid may be recirculated from the bioreactor to a homogenizer. In some embodiments, the bioreactor may be in fluid communication with the filter. In some embodiments, a cellular lysate may flow from the bioreactor to the filter.

In some embodiments, the bioreactor may be used only for cell culture but not the cell lysing process and a cell lysing process is carried out in a separate cell lysing instrument, such as a cell lysing vessel. In some embodiments, the cell lysing device disclosed herein may be in fluid communication with the filter disclosed herein. In some embodiments, the cell lysing vessel may comprise a container suitable for the cell lysing process disclosed herein. In some embodiments, the cell lysing vessel may be selected based on the volume of the cell culture, the volume of the harvested cells, or the volume of the resuspended cells, that are used in the CFPS method disclosed herein. In some embodiments, the cell lysing vessel may comprise a tank, a test tube, a beaker, a flask, a multi-well plate, or a combination thereof. In some embodiments, the cell lysing device may comprise a homogenizer, a sonicator, a heat exchanger, a nitrogen cavitation device, a freeze-thaw device, an acoustic lysis device, a bead-beating system, a syringe, or a combination thereof. In some embodiments, the lysing device may be operatively coupled to the lysing vessel to produce a cell lysate.

In some embodiments, the system may comprise a device configured for concentrating a cell culture used for preparation of CFPS disclosed herein. In some embodiments, the device for concentrating a cell culture may comprise a centrifuge, a tangential flow filtration device, a hollow-fiber filtration apparatus, a membrane separation device, or a combination thereof. In some embodiments, the membrane separation device may comprise a woven membrane, a non-woven membrane, an electrospun membrane, a hollow fiber membrane, a rolled sheet membrane, a flat sheet membrane, or a combination thereof.

In some embodiments, the system may comprise a reservoir. In some embodiments, the reservoir may contain a suspension medium configured to supply the medium to the cell lysing device of the system. In some embodiments, the reservoir may contain a suspension medium configured to supply the medium to a device for resuspending cells inside the lysis device. In some embodiments, the reservoir may contain a suspension medium configured to supply the medium to the cell lysing device disclosed herein. In some embodiments, the device for resuspending cells inside the lysis device may comprise a device for mixing the cells.

In some embodiments, one component of the system disclosed herein may comprise an inlet. In some embodiments, one component of the system disclosed herein may comprise an outlet. In some embodiments, one component of the system may be in fluid communication with another component of the system. In some embodiments, one component of the system may be in fluid communication with at least another component of the system. In some embodiments, two components of the system disclosed herein may both comprise an inlet and an outlet and are in fluid communication with each other so that a fluid may flow from the outlet of one component to the inlet of the other component.

In some embodiments, the system may comprise a post-clarification device to process the clarified cellular extract. In some embodiments, the system may comprise an incubator to carry out a run-off reaction. In some embodiments, the system may comprise an incubator to verify the sterility of the clarified cellular extract. In some embodiments, the system may comprise a membrane device. In some embodiments, the membrane device may be used for desalting or dialysis. In some embodiments, the membrane device may comprise a tangential flow filtration (TFF) device, a hollow fiber, a dialysis membrane, or a combination thereof.

In some embodiments, the system disclosed herein may be configured to deliver the clarified cellular extract to a reactor vessel for CFPS. In some embodiments, the system may be configured for flash freezing or freeze-drying the clarified cellular extract. In some embodiments, the system may be configured for storing the clarified cellular extract. In some embodiments, the system may be configured for storing the clarified cellular extract in liquid nitrogen. In some embodiments, the system may be configured for storing the clarified cellular extract at a temperature between about −200° C. and 25° C., e.g., about −200° C., −199° C., −198° C., −197° C., −196° C., −195° C., −194° C., −193° C., −192° C., −191° C., −190° C., −189° C., −188° C., −187° C., −186° C., −185° C., −184° C., −183° C., −182° C., −181° C., −180° C., −179° C., −178° C., −177° C., −176° C., −175° C., −174° C., −173° C., −172° C., −171° C., −170° C., −169° C., −168° C., −167° C., −166° C., −165° C., −164° C., −163° C., −162° C., −161° C., −160° C., −159° C., −158° C., −157° C., −156° C., −155° C., −154° C., −153° C., −152° C., −151° C., −150° C., −149° C., −148° C., −147° C., −146° C., −145° C., −144° C., −143° C., −142° C., −141° C., −140° C., −139° C., −138° C., −137° C., −136° C., −135° C., −134° C., −133° C., −132° C., −131° C., −130° C., −129° C., −128° C., −127° C., −126° C., −125° C., −124° C., −123° C., −122° C., −121° C., −120° C., −119° C., −118° C., −117° C., −116° C., −115° C., −114° C., −113° C., −112° C., −111° C., −110° C., −109° C., −108° C., −107° C., −106° C., −105° C., −104° C., −103° C., −102° C., −101° C., −100° C., −99° C., −98° C., −97° C., −96° C., −95° C., −94° C., −93° C., −92° C., −91° C., −90° C., −89° C., −88° C., −87° C., −86° C., −85° C., −84° C., −83° C., −82° C., −81° C., −80° C., −79° C., −78° C., −77° C., −76° C., −75° C., −74° C., −73° C., −72° C., −71° C., −70° C., −69° C., −68° C., −67° C., −66° C., −65° C., −64° C., −63° C., −62° C., −61° C., −60° C., −59° C., −58° C., −57° C., −56° C., −55° C., −54° C., −53° C., −52° C., −51° C., −50° C., −49° C., −48° C., −47° C., −46° C., −45° C., −44° C., −43° C., −42° C., −41° C., −40° C., −39° C., −38° C., −37° C., −36° C., −35° C., −34° C., −33° C., −32° C., −31° C., −30° C., −29° C., −28° C., −27° C., −26° C., −25° C., −24° C., −23° C., −22° C., −21° C., −20° C., −19° C., −18° C., −17° C., −16° C., −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C., or any temperature therebetween. In some embodiments, the system may comprise an instrument for isolating or extracting the polypeptide produced through CFPS.

In some embodiments, a component of the system disclosed herein may utilize a process equipment unit. In some embodiments, the process equipment unit may comprise a pump, a valve, a sensor, or a device holder for controlling the flow of a fluid including a cellular lysate and cellular extract, or a combination thereof. In some embodiments, the pump may comprise a vacuum pump or a peristaltic pump. In some embodiments, the system may be configured for continuous fluid communication among two or more components of the system. In some embodiments, the system may be configured so that a fluid may flow directly through each component of the system without interruption. In some embodiments, the system may comprise one or more selected from the group comprising a valve, a sensor, a detector, a surge tank and an equipment for any in-line solution changes so that the flow of a fluid inside the system may be temporarily interrupted to replace or remove a particular unit operation.

In some embodiments, the system may comprise a sensor and/or a probe for controlling and/or monitoring a process parameter of the system. In some embodiments, the process parameter may comprise temperature, pressure, pH, conductivity, dissolved oxygen (DO), dissolved carbon dioxide, mixing rate, flow rate, any other product parameter, or a combination thereof. In some embodiments, the sensor may comprise an optical sensor. In some embodiments, the system may be configured so that process control is achieved in a manner that does not compromise the sterility of the system. In some embodiments, the senor and/or probe may be connected to a sensor electronics module so that the output of the sensor and/or probe is sent to a terminal board and/or a relay box. In some embodiments, the result of a sensing operation may be input into a computer-implemented control system for calculation and control of various parameters, including but not limited to, temperature, weight/volume measurements and purity, and for display and user interface. In some embodiments, the control system may comprise one or more selected from an electronic system, a mechanical system, a pneumatic system, for controlling at least one process parameter. It is obvious to one skilled in the art that the control system may perform other functions and the embodiments disclosed herein do not, in any manner, limit the control system to any particular function or set of functions.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Cell-Free Polypeptide Synthesis Using Cellular Lysate Clarified Via Filtration

This example illustrates a method for preparing crude cellular extract clarified through filtration instead of the conventional method of clarification through centrifugation. This example demonstrated that a clarification method via filtration results in a substantial yield of protein produced from cell-free synthesis reaction.

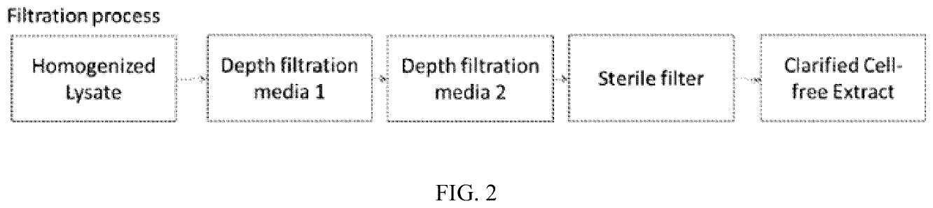

Cells were cultured, harvested, and lysed according to conventional methods, as illustrated in FIG. 1. Results of cell-free polypeptide synthesis were compared between cellular extract clarified via centrifugation and cellular extract clarified via filtration. Cellular extracts were clarified via centrifugation to produce an E. coli extract as previously reported in E. coli extract preparation protocols. The cellular extract clarified via filtration was prepared following the process illustrated in FIG. 2. Briefly, a peristaltic pump was used to drive the flow of cellular extract through the filtration medium. The homogenized cellular lysate was filtered through a first depth filter (15 μm-0.5 μm) to produce a first clarified cellular lysate. Then the first clarified cellular lysate was filtered through a second depth filter (0.5 μm-0.2 μm) to produce a second clarified cellular lysate. Last, the second clarified cellular lysate was filtered through a sterile filter (0.2 μm) to produce the clarified cellular extract for the cell-free polypeptide synthesis reaction.

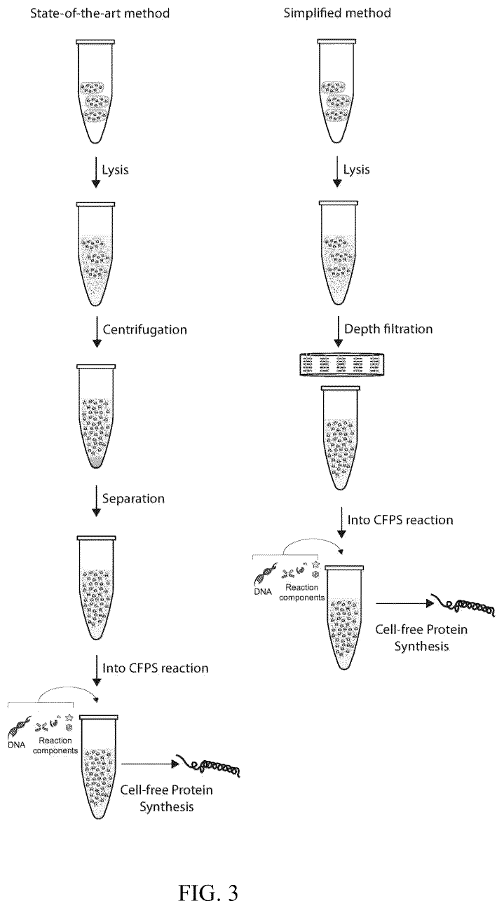

Cellular extracts clarified via centrifugation and cellular extracts clarified via filtration were then used for cell-free polypeptide synthesis based on the processes illustrated in FIG. 3. A DNA template encoding a superfolder green fluorescent protein (sfGFP) was added to both the cellular extract clarified via centrifugation and cellular extract clarified via filtration. The sfGFP DNA contained a T7 promoter and a strong ribosome binding site at the 5′ end of the coding sequence of sfGFP as well as a T7 terminator at the 3′ end of the coding sequence of sfGFP. The T7 promoter sequence was SEQ ID No: 1 and the T7 terminator sequence was SEQ ID No: 2. These regulatory elements ensured high levels of transcription and translation within the complex extract environment.

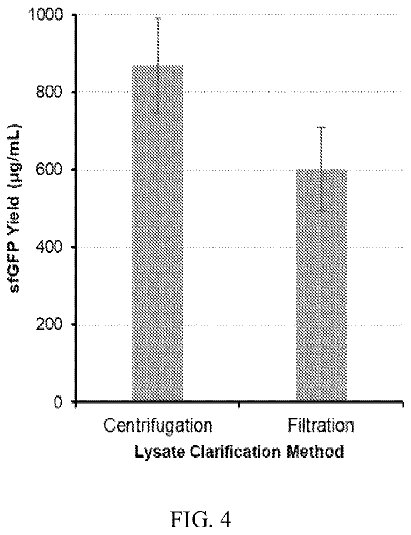

As shown in FIG. 4, the protein yield from cellular extract clarified via filtration was comparable to that from cellular extract clarified via centrifugation, as demonstrated by synthesis of sfGFP.

### Example 2: The Effect of Filter Pore Size on Protein Yield of CFPS

This example illustrates the effect of filter pore size on the cellular extract clarified via filtration and the proteins produced from CFPS using the filtered cellular extract.

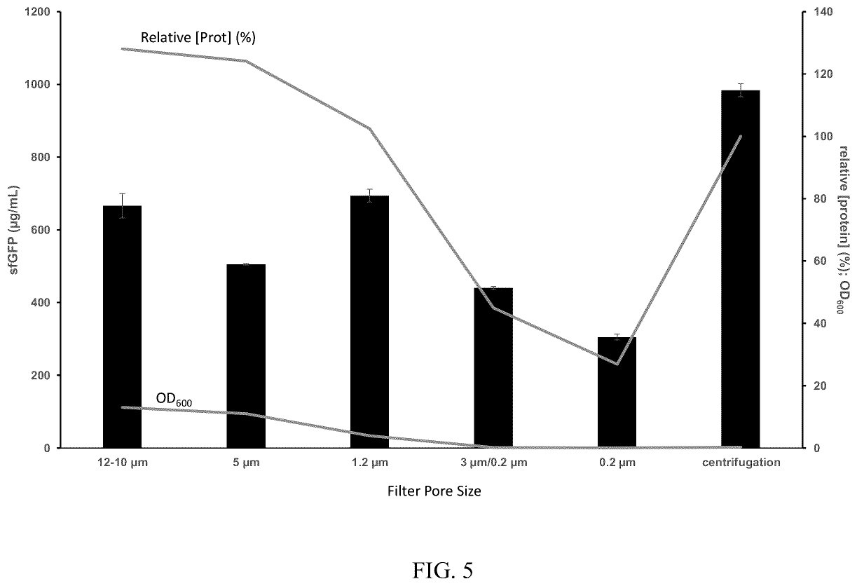

As illustrated in FIG. 5 and listed in Table 1, filters with a range of pore sizes, 12-10 microns, 5 microns, 1.2 microns, 3 microns/0.2 micron, and 0.2 micron, were used to filter the cellular lysate and assess their effects on clarified cellular extract activity and CFPS's overall tolerance. Compared to the state-of-the-art method for clarification of cellular lysate through centrifugation, filtration resulted in a decrease in cell-free polypeptide synthesis yield but was still tolerated. The cellular extract activity was related to its concentration of host cell protein and optical density. As filter pore sizes used in the filtration process decreased, the host cell protein concentration also decreased (line labeled with Relative [Prot]% (relative protein percentage)).

TABLE 1
Table 1: Values of protein concentration,
OD₆₀₀, and relative protein percentage (relative
[prot](%)) under different clarification methods

| Clarification | | sfGFP (μg/mL) | OD₆₀₀ | relative [prot] (%) |
|---|---|---|---|---|
| 12-10 | microns | 666.02 | 13 | 128.09 |
| 5 | microns | 505.06 | 11 | 124.12 |
| 1.2 | microns | 694.05 | 3.9 | 102.38 |
| 3/0.2 | micron | 440.41 | 0.11 | 44.96 |
| 0.2 | micron | 305.24 | 0.03 | 26.84 |

| | | | |
|---|---|---|---|
| Centrifugation | 984.12 | 0.22 | 100.00 |

The results, as listed in Table 1, indicated that filters were capturing host cell proteins, some of which may be utilized for CFPS. Similarly, optical density readings (OD₆₀₀) also decreased with smaller pore size (line labeled with OD₆₀₀), indicating further and better clarification. These two variables, i.e., extract protein concentration and extent of extract clarification, need to be balanced in order to produce a well-functioning cellular extract. Comparison of the activity of cellular extract prepared using the 3 microns/0.2 micron filter and the 0.2 micron filter indicated that a depth filtration train leading up to a 0.2 micron pore size would help retain some of those host cell proteins useful for CFPS while achieving a similar level of clarification.

### Example 3: The Effect of Filter Pore Size on Throughput and Average Flux on CFPS

This example illustrates the effect of filter pore sizes on the parameters of Throughput and Average Flux during the lysate clarification process.

Throughput, i.e., volume of lysate filtered per filter surface area, and Average Flux of filtrate, i.e., volume of lysate filtered per filter surface area per time, are two parameters related to the filtration-mediated clarification process of cellular lysate and could be utilized to optimize the filtration process. The following filters were used: 12-10 microns, 1.2 microns, 3 microns/0.2 micron, 0.2 micron.

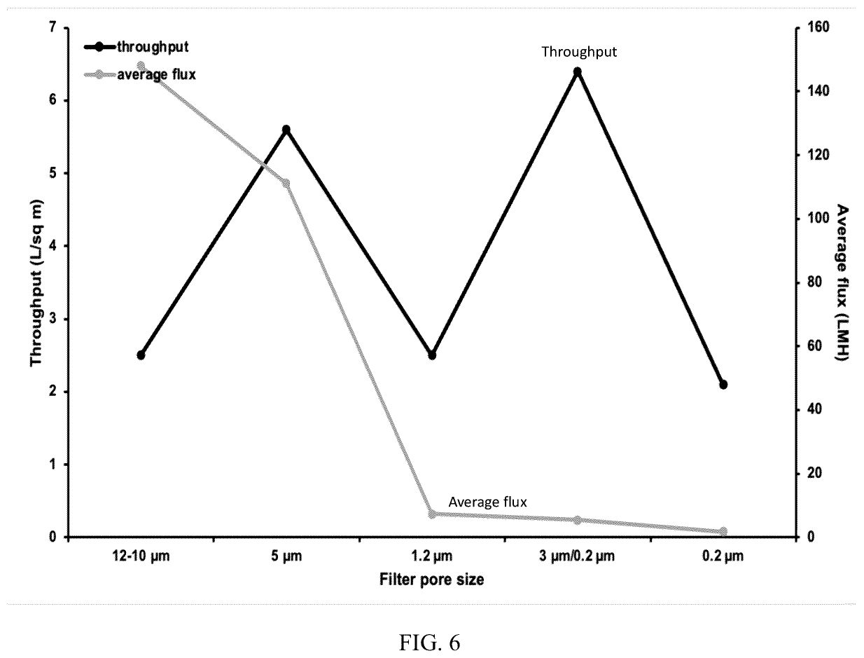

As illustrated in FIG. 6 and listed in Table 2, Throughput and Average Flux were measured in a series of filtration processes using different filter pore sizes.

TABLE 2
Table 2: Throughput and Average Flux values of clarification
methods using filters of different pore sizes

| | | |
|---|---|---|
| Pore size | Throughput | Average Flux |

| | | | |
|---|---|---|---|
| 12-10 | microns | 2.5 | 148.1 |
| 5 | microns | 5.6 | 111.1 |
| 1.2 | microns | 2.5 | 7.4 |
| 3/0.2 | micron | 6.4 | 5.5 |
| 0.2 | micron | 2.1 | 1.8 |

The driving force used during the filtration process was centrifugal force as opposed to monitored pressure, which may explain the inconsistency between the Throughput of the 12-10 microns filter group and the 5 microns filter group, since one may expect the 12-10 microns filter to have a Throughput greater than or similar to the 5-micron filter given their relatively large pore size. Average Flux, as expected, decreased as the pore size of the filter became smaller. The Throughput of the 3 microns/0.2 micron depth filter was greater than that of the 1.2 microns filter despite the fact that the 3 microns/0.2 micron filter ultimately pushes through a smaller pore size and has the ability to produce a better clarified extract. The 3 microns/0.2 micron depth filter also had a greater throughput than the 0.2 micron filter alone, which indicated that a depth filter or filter train with successively smaller pores may not only result in a more productive extract with similar extent of clarification but also a more efficient process that would not require as much time or filter area, which would therefore lower costs of operation.

### Example 4: The Effect of Filtration Trains on CFPS

This example illustrates the effect of filter types and filter pore sizes on CFPS.

To evaluate depth filtration trains, different depth filters, membrane filters, and combinations thereof into filter trains were used for clarification of cellular lysate for CFPS in order to assess their efficiency in an extract clarification process. The filters used in this example included the depth filters 0.2-3.0 microns, 0.4-15.0 microns, 6.0-30.0 microns, 0.2-0.8 micron, 0.1-0.3 micron, 0.2-0.5 micron, as well as several membrane filters with PES media or PES hybrid media.

For the purpose of lysate clarification, throughput and average filtrate constant flux were considered relevant metrics. In optimizing for these metrics, a train of two 0.4-15 microns depth filters followed by a 0.2-0.5 micron depth filter was found to have the greatest throughput as well as the greatest average filtrate constant flux. This train was found to be better than a 2×0.2-0.8 micron train, a 2×0.2-3.0 microns to 0.1-0.3 micron train, as well as a 6-30 microns to 0.2-3.0 microns train. With the depth filtration train selected, different membrane filters were tested, and a PES filter was found to have the highest throughput. The throughput metric was considered closely for scale-up purposes.

A modified version of the final filter train was used to confirm retained extract activity after the lysate clarification process. The modified filter train included a single 0.4-15 microns filter followed by 0.2-0.5 microns and a 0.2 micron PES membrane filter. The protein yield of cell-free extract filtered using the modified filter train was illustrated in FIG. 4, as discussed in Example 1 above.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for cell-free polypeptide synthesis comprising:
(a) lysing a cell to produce a cellular lysate;
(b) passing the cellular lysate through a filter to produce a cellular extract;
(c) contacting the cellular extract with a nucleic acid encoding a polypeptide to produce a reaction mixture; and
(d) incubating the reaction mixture to produce the polypeptide.

2. The method of claim 1, wherein any one or more of the cellular lysate, the cellular extract, or the reaction mixture are not subject to clarification through centrifugation.

3-4. (canceled)

5. The method of claim 1, further comprising flocculating or precipitating, or both flocculating and precipitating, the cellular lysate before the passing through the filter.

6. (canceled)

7. The method of claim 1, wherein the filter comprises a surface filter or a depth filter.

8. (canceled)

9. The method of claim 1, wherein the passing comprises one or more selected from the group comprising cross-flow filtration, belt filtration, microfiltration, ultrafiltration, microporous filtration, vacuum-drum filtration, sieving, membrane filtration, sand filtration, and screen filtration.

10. The method of claim 1, wherein the filter comprises a pore that retains or rejects a particle with a size of more than about 0.1 microns to more than about 100 microns.

11-24. (canceled)

25. The method of claim 1, wherein the filter comprises at least two filters that are arranged in series or in parallel, or wherein the filter comprises at least two filters that are arranged in series with gradually decreasing pore sizes.

26. (canceled)

27. The method of claim 25, wherein the filter comprises:
(a) a first filter comprising a pore that retains or rejects a particle with a size between about 0.5 micron and about 15 microns;
(b) a second filter comprising a pore that retains or rejects a particle with a size between about 0.2 micron and about 0.5 micron; and
(c) a third filter comprising a pore that retains or rejects a particle with a size of about 0.2 micron.

28-39. (canceled)

40. The method of claim 1, further comprising obtaining a cell from a cell culture.

41. The method of claim 40, wherein the obtaining comprises subjecting the cell culture to one or more selected from the group comprising centrifugation, tangential flow filtration, membrane separation, and a combination thereof.

42. (canceled)

43. The method of claim 1, wherein the lysing comprises one or more selected from the group comprising sonication, homogenization, nitrogen cavitation, freeze-thawing, syringing, chemical lysis, enzymatic lysis, osmotic lysis, French press lysis, bead-beating, and a combination thereof.

44. The method of claim 1, further comprising freezing or freeze-drying the cellular extract.

45. (canceled)

46. The method of claim 40, wherein the cell comprises a transgenic cell, a mammalian cell, a bacterial cell, a plant cell, a yeast cell, an insect cell, a fungal cell, an algal cell, or a combination thereof.

47-50. (canceled)

51. The method of claim 1, wherein the nucleic acid comprises DNA comprising a promoter and a terminator located downstream of the 3′ end of an open reading frame.

52. (canceled)

53. The method of claim 51, wherein the promoter comprises a T7 promoter comprising a sequence having at least 80% sequence identity with SEQ ID No: 1, and wherein the terminator comprises a T7 terminator comprising a sequence having at least 80% sequence identity with SEQ ID No: 2.

54-65. (canceled)

66. The method of claim 1, wherein the reaction mixture comprises an amino acid, a nucleotide, a salt, a cofactor, an energy source, a translation template, or a combination thereof.

67-69. (canceled)

70. The method of claim 66, wherein the energy source comprises one or more or glutamate, pyruvate, or glucose, and is present at a concentration between about 10 mM and about 400 mM.

71-72. (canceled)

73. The method of claim 66, wherein the salt comprises potassium at a concentration between about 50 mM and about 500 mM, or magnesium at a concentration between about 1 mM and about 30 mM, or ammonium at a concentration between about 1 mM and about 400 mM.

74-76. (canceled)

77. The method of claim 1, further comprising isolating the polypeptide from the reaction mixture.

78. (canceled)

79. The method of claim 66, wherein the nucleotide comprises a nucleoside monophosphate (NMP), a nucleoside diphosphate (NDP), a nucleoside triphosphate (NTP), or a combination thereof.

80. (canceled)
